SEQUENCE LISTING (39 sequences)

SEQ ID NO: 1 - protein, 5 aa, Artificial
  Linker
DGGGS

SEQ ID NO: 2 - protein, 5 aa, Artificial
  Linker
TGEKP

SEQ ID NO: 3 - protein, 9 aa, Artificial
  Linker
LRQKDGERP

SEQ ID NO: 4 - protein, 4 aa, Artificial
  Linker
GGRR

SEQ ID NO: 5 - protein, 5 aa, Artificial
  Linker
GGGGS

SEQ ID NO: 6 - protein, 8 aa, Artificial
  Linker
GGRRGGGS

SEQ ID NO: 7 - protein, 9 aa, Artificial
  Linker
LRQRDGERP

SEQ ID NO: 8 - protein, 12 aa, Artificial
  Linker
LRQKDGGGSERP

SEQ ID NO: 9 - protein, 16 aa, Artificial
  Linker
LRQKDGGGSGGGSERP

SEQ ID NO: 10 - DNA, 17 bp, Artificial
  AnxA8 forward primer
acgcgcagtgccactca

SEQ ID NO: 11 - DNA, 22 bp, Artificial
  AnxA8 Reverse primer
tgatgctgtcctcaatgctctt

SEQ ID NO: 12 - DNA, 35 bp, Artificial
  AnxA8 probe
ctgagagtgtttgaagagtatgagaaaattgccaa

SEQ ID NO: 13 - DNA, 16 bp, Artificial
  Kip2 forward primer
gcgcggcgatcaagaa

SEQ ID NO: 14 - DNA, 18 bp, Artificial
  Kip2 reverse primer
acatcgcccgacgacttc

SEQ ID NO: 15 - DNA, 24 bp, Artificial
  Kip2 probe
ccgggcctctgatctccgatttct

SEQ ID NO: 16 - DNA, 17 bp, Artificial
  GRP forward primer
aggccctgggcaatcag

SEQ ID NO: 17 - DNA, 25 bp, Artificial
  GRP reverse primer
caactttgccttttgaacctacatc

SEQ ID NO: 18 - DNA, 28 bp, Artificial
  GRP probe
agccttcgtgggattcagaggatagcag

SEQ ID NO: 19 - DNA, 21 bp, Artificial
  GAPDH forward primer
ccatgttcgtcatgggtgtga

SEQ ID NO: 20 - DNA, 20 bp, Artificial
  GAPDH reverse primer
catggactgtggtcatgagt

SEQ ID NO: 21 - DNA, 24 bp, Artificial
  GAPDH probe
tcctgcaccaccaactgcttagca

SEQ ID NO: 22 - DNA, 61 bp, Artificial
  a segment containing the Kozak translation initiation sequence and the SV40 nuclear localization sequence
gaattcgctagcgccaccatggcccccaagaagaagaggaaggtgggaatccatggggtac

SEQ ID NO: 23 - DNA, 187 bp, Artificial
  a segment containing a BamHi site, the KRAB-A box from KOX1, the FLAG epitope, and a HindIII site
ggtacccggggatcccggacactggtgaccttcaaggatgtatttgtggacttcaccagggaggagtggaagctgctggacactgctcagcagatcgtgtacagaaatgtgatgctggagaactataagaacctggtttccttgggcagcgactacaaggacgacgatgacaagtaagcttctcgag

SEQ ID NO: 24 - DNA, 10 bp, Artificial
  ZFP 734 kip2 binding site
ggggctgggt

SEQ ID NO: 25 - protein, 7 aa, Artificial
  ZFP 734 kip2 F1 sequence
RSDHLAR

SEQ ID NO: 26 - protein, 7 aa, Artificial
  ZFP 734 kip2 F2 sequence
QSSDLSR

SEQ ID NO: 27 - protein, 7 aa, Artificial
  ZFP 734 kip2 F3 sequence
RSDHLSR

SEQ ID NO: 28 - DNA, 9 bp, Artificial
  ZFP 1727 GRP binding site
gtggggagg

SEQ ID NO: 29 - protein, 7 aa, Artificial
  ZFP 1727 GRP F1 sequence
RSDNLAR

SEQ ID NO: 30 - protein, 7 aa, Artificial
  ZFP 1727 GRP F2 sequence
RSDHLTR

SEQ ID NO: 31 - protein, 7 aa, Artificial
  ZFP 1727 GRP F3 sequence
TSGHLVR

SEQ ID NO: 32 - DNA, 10 bp, Artificial
  ZFP 757 anxA8 binding site
cgggcggctg

SEQ ID NO: 33 - protein, 7 aa, Artificial
  ZFP 757 anx A8 F1 sequence
QSSDLRR

SEQ ID NO: 34 - protein, 7 aa, Artificial
  ZFP 757 anx A8 F2 sequence
RSDELQR

SEQ ID NO: 35 - protein, 7 aa, Artificial
  ZFP 757 anx A8 F3 sequence
RSDHLRE

SEQ ID NO: 36 - DNA, 10 bp, Artificial
  ZFP 993 kip2 binding site
ggggctgggt

SEQ ID NO: 37 - protein, 7 aa, Artificial
  ZFP 993 kip2 F1 sequence
RSDHLAR

SEQ ID NO: 38 - protein, 7 aa, Artificial
  ZFP 993 kip2 F2 sequence
TSGELVR

SEQ ID NO: 39 - protein, 7 aa, Artificial
  ZFP 993 kip2 F3 sequence
RSDHLSR

(19) United States
(12) Patent Application Publication
(10) Pub. No.: US 2004/0235002 A1
(43) Pub. Date: Nov. 25, 2004

(54) Multiplex screening assays

(75) Inventors: Michael Holmes, Oakland, CA (US); Christin Tse, Davis, CA (US)

Correspondence Address: ROBINS & PASTERNAK, 1731 EMBARCADERO ROAD, SUITE 230, PALO ALTO, CA, 94303, US

(73) Assignee: Sangamo BioSciences, Inc., Richmond, CA (US)

(21) Appl. No.: 10/666,923

(22) Filed: Sep. 18, 2003

Related U.S. Application Data
(60) Provisional application No. 60/412,345, filed on Sep. 20, 2002.

(51) Int. Cl. C12Q 1/68; G01N 33/53; G01N 33/567

(52) U.S. Cl. 435/6; 435/7.2

(57) ABSTRACT

Disclosed herein are methods and compositions for multiplex screening. A functional domain of a drug target is fused to a zinc finger protein (ZFP) binding domain targeted to an endogenous reporter gene. Expression of the reporter gene provides an assay for the activity of the functional domain and, hence for agonists and antagonists of the functional domain. Moreover, a plurality of functional domain-ZFP fusions can be introduced into a single cell line, allowing simultaneous assay of all of the functional domains. Besides being obtained from a drug target, a functional domain can be obtained from, for example, a protein related to the drug target, a protein involved in drug metabolism and/or a protein involved in drug toxicity.

### CROSS-REFERENCE TO RELATED APPLICATIONS

This application claims the benefit of U.S. Provisional Application Ser. No. 60/412,345, filed Sep. 20, 2002, which application is hereby incorporated by reference in its entirety.

### TECHNICAL FIELD

The present disclosure is in the field of screening assays; for example, screens for agonists and antagonists of nuclear hormone receptors. More particularly, improved methods and compositions for drug discovery and lead optimization are provided.

### BACKGROUND

The process of discovering a new therapeutic traditionally involves the following stages: (1) identification of a drug target, (2) validation of the target, (3) screening for compounds that affect the activity of the target, (4) testing lead compounds for toxicity, (5) testing lead compounds for side effects, and (6) examining the metabolism and stability of lead compounds, in the patient or in an appropriate model system.

Once a potential therapeutic target has been identified and validated, the initial stage of drug discovery requires the screening of often hundreds or thousands of compounds to identify those that regulate the target in the appropriate therapeutic manner. This screening process requires the development of assays that can rapidly and inexpensively measure the potency of compounds to regulate the target factor of interest. These high throughput screening assays can take many forms that include either cell-based or in vitro biochemical assays that rely on colorimetric, fluorescence, or luminescence-based detection assays that measure RNA or protein abundance, enzymatic activity, or the physical interaction of proteins to form a functional complex. See, for example, Mere, L., et al., Miniaturized FRET assays and microfluidics: key components for ultra-high-throughput screening. Drug Discov Today, 1999. 4(8): p. 363-369; Warrior, U., et al., Application of QuantiGene nucleic acid quantification technology for high throughput screening. J Biomol Screen, 2000. 5(5): p. 343-52; and Mendoza, L. G., et al., High-throughput microarray-based enzyme-linked immunosorbent assay (ELISA). Biotechniques, 1999. 27(4): p. 778-80, 782-6, 788.

A constant challenge facing the drug discovery field is to increase the speed and efficiency by which potential lead compounds are identified, from the thousands of chemical compounds tested in compound library screens, and optimized into potent drugs. A common problem encountered in lead optimization is that a compound originally identified by virtue of its ability to modulate the activity of one or a few specific target proteins also often has one or more deleterious side effects. Detrimental effects can be caused by the lack of specificity of a compound, causing the compound to target a broad range of factors and biological processes, in addition to the intended target. Other areas of concern include drug toxicity and metabolism. Compounds that elicit toxic responses can disrupt normal cellular and tissue function and/or lead to cell death. Certain compounds have also been demonstrated to regulate their own metabolism, stimulating their breakdown and removal from the body, leading to decreased drug efficacy. See, e.g., Willson, T. M. and S. A. Kliewer, PXR, CAR and drug metabolism. Nat Rev Drug Discov, 2002. 1(4): p. 259-66. Screening technologies that could integrate analyses of compound efficacy, specificity, and toxicity in a single high throughput assay would greatly increase the speed and efficiency of drug development.

Current high throughput screening assays generally focus on measuring the effectiveness of compounds in regulating the activity of a single factor (the target), and rely on often extended processes of secondary screening and follow-up analyses to determine other characteristics of compound function, such as specificity and toxicity. This increases the amount of time and cost required to develop and optimize compounds into potent drugs with high therapeutic indices (i.e. high efficacy, high specificity, low toxicity), because analysis of side effects is conducted subsequent to the determination of the effect of a compound on the intended target. As a result, many compounds, originally selected because of their activity on the target, are eventually discarded because of subsequently discovered side effects, resulting in wasted time and effort devoted to “hits” which eventually prove to be unsatisfactory. Accordingly, there is a need for screening methods that reduce the time and expense spent on identifying side effects of active compounds.

Thus, the processes of drug discovery and lead optimization could be made faster, more efficient, and less expensive with the creation of a screening assay that provided simultaneous information on various compound characteristics (i.e. efficacy, specificity, toxicity, and drug metabolism).

Simultaneous monitoring of multiple reporters (i.e., multiplexing) is one way in which it might be possible to determine efficacy of a compound, while at the same time, examining e.g., possible side effects and metabolism. However, the technology to support multiplex assays for high throughput screening has been slow to develop. Although assay systems capable of measuring the abundance of greater than 10 different proteins and/or RNA species in a single sample are available (e.g., Luminex Tech., Aclara eTag, and High Throughput Genomics ArrayPlate), their use in a multiplex platform is limited by the dearth of well-characterized reporter genes. For example, although the reporter gene encoding green fluorescent protein (GFP) has been modified to generate several additional colors, fluorescent detection capability limits the number of fluorescent proteins that it is possible to assay in a single cell line to three colors.

Thus, for a useful multiplex assay, it would be desirable to have multiple reporter readouts, preferably in the form of cellular genes. However, there is at present a limited ability to specifically and uniquely target proteins to different reporter genes in a single cell line via natural DNA-binding domains.

A particularly severe problem, in this regard, accompanies assays for members of the nuclear hormone receptor superfamily, since many of these factors share identical or similar DNA-binding specificities, causing them to bind to and compete for the same DNA binding sequences. See, for example, Aranda, A. and A. Pascual, Nuclear hormone receptors and gene expression. Physiol Rev, 2001. 81(3): p. 1269-304; Kraus, R. J., et al., Estrogen-related receptor alpha 1 actively antagonizes estrogen receptor-regulated transcription in MCF-7 mammary cells. J Biol Chem, 2002. 277(27): p. 24826-34; and Burbach, J. P., et al., Repression of estrogen-dependent stimulation of the oxytocin gene by chicken ovalbumin upstream promoter transcription factor I. J Biol Chem, 1994. 269(21): p. 15046-53. Thus, for example, a reporter gene intended to be regulated through an upstream estrogen receptor binding site, besides being regulated by ER, is also likely to be regulated by one or more estrogen-related receptors (ERRs) and/or the COUP-TF receptor. The same problem can occur with identifying compounds that selectively regulate one member of a family of different protein isotypes or splice variants, since the DNA-binding characteristics of each of these factors can be identical or extremely similar.

One attempt to overcome this problem is to fuse a drug target (e.g., a nuclear receptor or related factor) to a heterologous DNA-binding domain, such as the DNA-binding domain from the yeast protein GAL4, and insert a GAL4 binding site upstream of the reporter gene. See, for example, WO 95/18380. However, it remains difficult to conduct multiplex assays using this strategy, because only a few such well-characterized DNA-binding domains are available (e.g., GAL4, LexA). It also becomes difficult to rapidly generate screening cell lines that have multiple reporter constructs stably or transiently expressed in them.

WO 01/21215 discloses an assay in which an exogenous transcription factor is targeted to an endogenous reporter gene, which can be used to measure effects of compounds on the exogenous transcription factor. However, it does not disclose or suggest a multiplex assay in which a plurality of endogenous genes are targeted by exogenous molecules.

Multiplex assays are disclosed in U.S. Pat. No. 6,410,245; WO 98/48274, WO 98/53093, WO 98/58074 and WO 01/75443. However, none of these assays involve the use of zinc finger proteins targeted to endogenous reporter genes.

Yet another problem with current screening assays is that a compound can often regulate the activity or expression of a reporter gene through a mechanism independent of the intended target, creating noise in the assay that is required to be filtered out in later studies. Another disadvantage of current methods for high throughput screening is that the amount of compound available for primary and secondary screening purposes is often very limited, making it difficult to conduct multiple screens with different factors and/or perform follow-up testing.

Thus, the fields of drug discovery and lead optimization would be advanced by the availability of high-throughput assays capable of simultaneously characterizing several properties of a drug, such as, for example, efficacy, specificity, toxicity and metabolic properties. Additionally, methods and compositions for rapid characterization of the specificity of a compound for a molecular target, especially in the presence of related molecules, would advance the field. Furthermore, methods to confirm that changes in the regulation of a reporter by a compound are the result of interaction of the compound with its molecular target are needed. Finally, screening methods that are effective with smaller amounts of compound would be beneficial.

### SUMMARY

Disclosed herein are compositions and methods useful in multiplex assays for compound screening, comprising fusions between a functional domain and an engineered zinc finger protein, in which the engineered zinc finger protein is targeted to an endogenous reporter gene. Thus, one or more endogenous cellular genes serve as readout for the activity of the functional domain(s), as well as the effect of a compound on the activity of the functional domain. The disclosed assay methods and compositions can be used to screen a compound e.g., for specificity, toxicity or metabolic properties.

In certain embodiments, the disclosure provides a method for screening a compound, wherein the method comprises contacting the compound with a cell, wherein the cell comprises:

(i) a first polynucleotide encoding a protein comprising a fusion between a first functional domain and a first engineered zinc finger protein targeted to a first endogenous cellular gene; and

(ii) a second polynucleotide encoding a protein comprising a fusion between a second functional domain and a second engineered zinc finger protein targeted to a second endogenous cellular gene; and measuring expression of the first and second endogenous genes.

In other embodiments, described herein is a method for determining the effect of a compound on the activity of a functional domain, comprising the steps of: (a) contacting the compound with a cell, wherein the cell comprises: (i) a first polynucleotide encoding a protein comprising a fusion between a first functional domain (e.g., drug target or functional fragment thereof) and a first engineered zinc finger protein targeted to a first endogenous cellular gene; and (ii) a second polynucleotide encoding a protein comprising a fusion between a second functional domain (e.g., drug target, functional fragment thereof, a protein related to the drug target or functional fragment thereof) and a second engineered zinc finger protein targeted to a second endogenous cellular gene; and (b) measuring expression levels of the first and second genes as compared to cells not contacted with the compound, thereby determining the effect of the compound on the activity of the functional domain.

In certain embodiments, the first and second functional domains are from the same drug target while in other embodiments, the first and second functional domains are from different drug targets. The first and/or second functional domain(s) may be, for example, a xenobiotic receptor or functional fragment thereof; a molecule involved in drug metabolism or a functional fragment thereof; a hormone receptor or a functional fragment thereof; and/or an orphan receptor or a functional fragment thereof. The first and/or second polynucleotides may be stably integrated into the chromosome of the cell (e.g., mammalian cell).

In any of the methods described herein, expression of the endogenous genes can be measured by assaying RNA levels, protein levels, and/or enzymatic activity of the gene products. Further, in any of the methods described herein, expression of the first endogenous gene may be modulated (e.g., activated or repressed) by the first functional domain. In any of the methods, specificity, toxicity and/or the effect of the compound on metabolic processes can be determined.

In certain embodiments, the first and/or the second functional domain is a drug target or functional fragment thereof. In these embodiments, the first and second functional domains can be from the same drug target or from different drug targets.

In additional embodiments, the first functional domain is obtained from a drug target and the second functional domain is obtained from a protein that is related to the drug target (e.g., a family member or splice variant); the first functional domain is obtained from a drug target and the second functional domain is obtained from a xenobiotic receptor; or the first functional domain is obtained from a drug target and the second functional domain is obtained from a protein that is involved in drug metabolism.

Exemplary sources of functional domains are hormone receptors and orphan receptors, or functional fragments thereof.

In certain embodiments, polynucleotides encoding fusions between a functional domain and an engineered zinc finger protein are stably integrated into a chromosome of a cell. Cells can be prokaryotic or eucaryotic, e.g., fungal, plant, insect or any type of animal cell, including but not limited to piscine, avian, ovine, equine, bovine, feline, canine, primate and human.

A fusion protein, as disclosed herein, is able to regulate expression of an endogenous gene in a cell. Regulation can be in the form of either activation or repression. Endogenous gene expression is measured by assaying RNA levels, protein levels and/or enzymatic activity of one or more gene products.

Also provided are cells comprising a first polynucleotide encoding a protein comprising a fusion between a first functional domain and a first engineered zinc finger protein targeted to a first endogenous cellular gene; and a second polynucleotide encoding a protein comprising a fusion between a second functional domain and a second engineered zinc finger protein targeted to a second endogenous cellular gene. In additional embodiments, cells can comprise third, fourth, fifth, etc. polynucleotides, each of which encodes a third, fourth, fifth, etc. fusion between a third, fourth, fifth, etc. functional domain and a third, fourth, fifth, etc. engineered zinc finger protein targeted to a third, fourth, fifth, etc. endogenous cellular gene.

In certain embodiments, the first and/or the second functional domain is a drug target or functional fragment thereof. In these embodiments, the first and second functional domains can be from the same drug target or from different drug targets. Similarly, third, fourth, fifth, etc. functional domains can be obtained from a drug target, and they can be the same or different from the drug target(s) from which first and/or second functional domains are obtained.

In additional embodiments, the first functional domain is obtained from a drug target and one or more of the second, third, fourth, fifth, etc. functional domains is obtained from a protein that is related to the drug target (e.g., a family member or splice variant); the first functional domain is obtained from a drug target and one or more of the second, third, fourth, fifth, etc. functional domains is obtained from a xenobiotic receptor; or the first functional domain is obtained from a drug target and one or more of the second, third, fourth, fifth, etc. functional domain is obtained from a protein that is involved in drug metabolism.

Exemplary sources of functional domains are hormone receptors and orphan receptors, or functional fragments thereof.

In certain embodiments, polynucleotides encoding fusions between a functional domain and an engineered zinc finger protein are stably integrated into a chromosome of the cell. Cells can be prokaryotic or eucaryotic, e.g., fungal, plant, insect or any type of animal cell, including but not limited to piscine, avian, ovine, equine, bovine, feline, canine, primate and human.

### BRIEF DESCRIPTION OF THE DRAWINGS

FIG. 1 is a schematic diagram showing the domain structure of a typical nuclear hormone receptor.

FIG. 2 is a schematic diagram showing the structure of a ZFP-LBD fusion as disclosed herein.

FIG. 3 shows the structure of the plasmid pcDNA3-modZFP-hFXR LBD (734-FXR LBD), which encodes a fusion of a kip2-targeted ZFP and a FXR ligand binding domain.

FIG. 4 shows the structure of the plasmid pcDNA3-modZFP-TRbeta (1727-TRb), which encodes a fusion of a GRP-targeted ZFP and a TRβ ligand binding domain.

FIG. 5 shows the structure of the plasmid pcDNA3-modZFP-hERalpha LBD (757-ERa), which encodes a fusion of an AnxA8-targeted ZFP and a ERα ligand binding domain.

FIG. 6 shows changes in the levels of mRNA expressed from the endogenous Kip2, GRP and AnxA8 genes in cells that had been transfected with three plasmids: one encoding a fusion between the FXR ligand-binding domain and a ZFP targeted to the Kip2 gene; one encoding a fusion between the TRβ ligand-binding domain and a ZFP targeted to the GRP gene; and one encoding a fusion between the ERα ligand-binding domain and a ZFP targeted to the Anx8 gene. The leftmost set of bars shows expression levels of the three genes in negative control cells (treated with DMSO). The second set of bars shows expression levels of the three genes in cells treated with β-estradiol. The third set of bars shows expression levels of the three genes in cells treated with T3. The fourth (rightmost) set of bars shows expression levels of the three genes in cells treated with CDCA. In each set of bars, the leftmost bar indicates levels of Kip2 mRNA, the center bar indicates levels of GRP mRNA, and the rightmost bar indicates levels of AnxA8 mRNA.

FIG. 7 shows levels of Kip2 and GRP mRNA in cells treated with different concentrations of β-estradiol. The cells contained an integrated construct expressing a Kip2-targeted ZFP binding domain fused to the ligand binding domain of ERα and a transfected construct expressing a GRP-targeted ZFP binding domain fused to the ligand-binding domain of TRβ. Fold change in RNA level (FC) compared to untreated cells is shown on the ordinate, and β-estradiol concentrations are given on the abscissa. “0” denotes cells treated with DMSO only. The upper line shows Kip2 mRNA levels; the lower line shows GRP mRNA levels.

FIG. 8 shows levels of Kip2 and GRP mRNA in cells treated with different concentrations of T3. The cells contained an integrated construct expressing a Kip2-targeted ZFP binding domain fused to the ligand binding domain of ERα and a transfected construct expressing a GRP-targeted ZFP binding domain fused to the ligand-binding domain of TRβ. Fold change in RNA level (FC) compared to untreated cells is shown on the ordinate, and T3 concentrations are given on the abscissa. “0” denotes cells treated with DMSO only. The upper line shows GRP mRNA levels; the lower line shows Kip2 mRNA levels.

FIG. 9 shows the structure of the plasmid pcDNA3-modZFP-hERbeta LBD (1727-ERb), which encodes a fusion of an GRP-targeted ZFP and a ERβ ligand binding domain.

FIG. 10 shows levels of kip2 and GRP mRNA, in response to α-estradiol and β-estradiol, in cells which stably express two exogenous proteins: a kip2-targeted ZFP fused to the ERα ligand binding domain and a GRP-targeted ZFP fused to the ERβ ligand binding domain.

### DETAILED DESCRIPTION

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Third edition, Cold Spring Harbor Laboratory Press, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

Definitions

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T. Thus, the term polynucleotide sequence is the alphabetical representation of a polynucleotide molecule. This alphabetical representation can be input into databases in a computer having a central processing unit and used for bioinformatics applications such as functional genomics and homology searching.

Chromatin is the nucleoprotein structure comprising the cellular genome. “Cellular chromatin” comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H₂B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

Typical “control elements” include, but are not limited to, transcription promoters, transcription enhancer elements, silencers, locus control regions, insulators, boundary elements, matrix attachment regions, replication origins, cis-acting transcription regulating elements (transcription regulators, e.g., a cis-acting element that affects the transcription of a gene, for example, a region of a promoter with which a transcription factor interacts to modulate expression of a gene), transcription termination signals, as well as polyadenylation sequences (located 3′ to the translation stop codon), sequences for optimization of initiation of translation (located 5′ to the coding sequence), translation enhancing sequences, and translation termination sequences. Transcription promoters can include inducible promoters (where expression of a polynucleotide sequence operably linked to the promoter is induced by an analyte, cofactor, regulatory protein, small molecule, drug, etc.), repressible promoters (where expression of a polynucleotide sequence operably linked to the promoter is repressed by an analyte, cofactor, regulatory protein, small molecule, drug, etc.), and constitutive promoters, which are characterized by a constant level of activity in the absence of inducing or repressing substances.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. When claiming sequences relative to sequences described herein, the range of desired degrees of sequence identity is approximately 80% to 100% and any integer value therebetween. Typically the percent identities between the disclosed sequences and the claimed sequences are at least 70-75%, preferably 80-82%, more preferably 85-90%, even more preferably 92%, still more preferably 95%, and most preferably 98% sequence identity to the reference sequence (i.e., the sequences disclosed herein).

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two DNA, or two polypeptide sequences are “substantially homologous” to each other when the sequences exhibit at least about 70%-75%, preferably 80%-82%, more preferably 85%-90%, even more preferably 92%, still more preferably 95%, and most preferably 98% sequence identity to each other, or to a reference sequence, over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning: A Practical Approach, editor, D. M. Glover (1985) Oxford; Washington, D.C.; IRL Press; Nucleic Acid Hybridization: A Practical Approach, editors B. D. Hames and S. J. Higgins (1985) Oxford; Washington, D.C.; IRL Press.

“Selective hybridization” of two nucleic acid fragments can be determined as described herein. The degree of sequence identity between two nucleic acid molecules affects the efficiency and strength of hybridization events between such molecules. A nucleic acid sequence that is partially identical to a target molecule will at least partially inhibit the hybridization of a completely identical sequence to the target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a duplex or “hybrid” molecule. A nucleic acid molecule that is capable of hybridizing selectively to a target sequence under “moderately stringent” hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization, where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well known to those of skill in the art. Hybridization stringency refers to the degree to which hybridization conditions disfavor the formation of duplexes containing mismatched nucleotides, with higher stringency correlated with a lower tolerance for mismatches. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as varying wash conditions. The selection of a particular set of hybridization conditions is conducted following standard methods in the art (see, for example, Sambrook, et al., supra).

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” is a protein or segment within a larger protein that binds DNA in a sequence-specific manner as a result of stabilization of protein structure through coordination of a zinc ion. The term “zinc finger DNA binding protein” is often abbreviated as “zinc finger protein” or “ZFP.”

A “designed” zinc finger protein is a protein not occurring in nature whose design/composition results principally from rational criteria. Rational criteria for design include application of substitution rules and computerized algorithms for processing information in a database storing information of existing ZFP designs and binding data. A “selected” zinc finger protein is a protein not found in nature whose production results primarily from an empirical process such as phage display. See e.g., U.S. Pat. No. 5,789,538; U.S. Pat. No. 6,007,988; U.S. Pat. No. 6,013,453; U.S. Pat. No. 6,140,081; U.S. Pat. No. 6,140,466; WO 95/19431; WO 96/06166 and WO 98/54311. Both designed and selected ZFPs are examples of “engineered” ZFPs.

The term “naturally-occurring” is used to describe an object that can be found in nature, as distinct from being artificially produced by humans. Examples include naturally-occurring zinc fingers (e.g., a zinc finger that is encoded by the genome of an organism, as opposed to having been designed or selected), and naturally-occurring zinc finger proteins (e.g., a protein comprising multiple zinc fingers wherein the sequence of the entire protein, including the sequence and location of the zinc fingers in the protein, is encoded by the genome of an organism). For the purposes of the present disclosure, a protein comprising a collection of naturally-occurring zinc fingers, which are not normally present together in a naturally-occurring ZFP and/or which are not present in the order in which they occur in a naturally-occurring ZFP, is not a naturally-occurring protein, but is considered to be a type of engineered ZFP.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically joined in cis and can be contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a transcriptional activation domain (or functional fragment thereof), the ZFP DNA-binding domain and the transcriptional activation domain (or functional fragment thereof) are in operative linkage if, in the fusion polypeptide, the ZFP DNA-binding domain portion is able to bind its target site and/or its binding site, while the transcriptional activation domain (or functional fragment thereof) is able to activate transcription.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one ore more amino acid or nucleotide substitutions. Methods for determining the function of a nucleic acid (e.g., coding function, ability to hybridize to another nucleic acid, binding to a regulatory molecule) are well known in the art. Similarly, methods for determining protein function are well known. For example, the DNA-binding function of a polypeptide can be determined, for example, by filter-binding, electrophoretic mobility-shift, or immunoprecipitation assays. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

“Specific binding” between, for example, a ZFP and a specific target site means a binding affinity (i.e, K_{d}) of at least 1×10⁶ M⁻¹.

A “fusion molecule” is a molecule in which two or more subunit molecules are linked, preferably covalently. The subunit molecules can be the same chemical type of molecule, or can be different chemical types of molecules. Examples of the first type of fusion molecule include, but are not limited to, fusion polypeptides (for example, a fusion between a ZFP DNA-binding domain and a nuclear hormone receptor ligand-binding domain) and fusion nucleic acids (for example, a nucleic acid encoding a ZFP-LBD fusion polypeptide). Examples of the second type of fusion molecule include, but are not limited to, a fusion between a triplex-forming nucleic acid and a polypeptide, and a fusion between aminor groove binder and a nucleic acid.

An “exogenous molecule” is a molecule that is not normally present in a cell, but can be introduced into a cell by one or more genetic, biochemical or other methods. Normal presence in the cell is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotien, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., protein or nucleic acid (e.g., an exogenous gene). For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous molecule” is one that is normally present in a particular cell at a particular developmental stage under particular environmental conditions. For example, an endogenous nucleic acid can comprise a chromosome, the genome of a mitochondrion, chloroplast or other organelle, or a naturally occurring episomal nucleic acid. Additional endogenous molecules can include endogenous genes and endogenous proteins, for example, transcription factors and components of chromatin remodeling complexes.

A “gene,” for the purposes of the present disclosure, includes a DNA region encoding a gene product (see below), as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

An “endogenous gene” is a gene that is native to a cell, which is in its normal genomic and chromatin context and which is not heterologous to the cell. Endogenous genes can be cellular, microbial or viral. Endogenous microbial and viral genes refer to genes that are part of a naturally-occurring microbial or viral genome in a microbially- or virally-infected cell. The microbial or viral genome can be extrachromosomal, or it can be integrated into the host chromosome(s).

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs that are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” and “augmentation of gene expression” refer to any process that results in an increase in production of a gene product. A gene product can be either RNA (including, but not limited to, mRNA, rRNA, tRNA, enzymatic RNA and structural RNA) or protein. Accordingly, gene activation includes those processes that increase transcription of a gene and/or translation of a mRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes that increase translation include those that increase translational initiation, those that increase translational elongation and those that increase mRNA stability. In general, gene activation comprises any detectable increase in the production of a gene product, preferably an increase in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more.

“Gene repression” and “inhibition of gene expression” refer to any process that results in a decrease in production of a gene product. A gene product can be either RNA (including, but not limited to, mRNA, rRNA, tRNA, enzymatic RNA and structural RNA) or protein. Accordingly, gene repression includes those processes that decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes that decrease translation include those that decrease translational initiation, those that decrease translational elongation and those that decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more.

“Modulation” of gene expression includes both gene activation and gene repression. Modulation can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP (see, e.g., Mistili & Spector, (1997) Nature Biotechnology 15:961-964); changes in signal transduction; changes in phosphorylation and dephosphorylation; changes in receptor-ligand interactions; changes in concentrations of second messengers such as, for example, cGMP, cAMP, IP₃, and Ca2⁺; changes in cell growth, changes in neovascularization, and/or changes in any functional effect of gene expression. Measurements can be made in vitro, in vivo, and/or ex vivo. Such functional effects can be measured by conventional methods, e.g., measurement of RNA or protein levels, measurement of RNA stability, and/or identification of downstream or reporter gene expression. Readout can be by way of, for example, chemiluminescence, fluorescence, calorimetric reactions, antibody binding, inducible markers, ligand binding assays; changes in intracellular second messengers such as cGMP and inositol triphosphate (IP₃); changes in intracellular calcium levels; cytokine release, and the like.

“Eucaryotic cells” include, but are not limited to, fungal cells (such as yeast), plant cells, animal cells, mammalian cells and human cells.

A “regulatory domain” or “functional domain” refers to a protein or a polypeptide sequence that performs a function in a cell. Exemplary functions include transcriptional modulation activity, drug metabolism, and binding of messenger molecules such as e.g., hormones. In one embodiment, a regulatory domain is covalently or non-covalently linked to a ZFP to modulate transcription of a gene of interest. Alternatively, a ZFP can act alone, without a regulatory domain, to modulate transcription. Furthermore, transcription of a gene of interest can be modulated by a ZFP linked to multiple regulatory domains. In addition, a regulatory domain can be linked to any DNA-binding domain having the appropriate specificity to modulate the expression of a gene of interest. Exemplary functional domains can be obtained from transcription factors, coactivators, corepressors, nuclear hormone receptors, xenobiotic receptors, and proteins involved in drug metabolism.

A “target site” or “target sequence” is a sequence that is bound by a binding protein or binding domain such as, for example, a ZFP. Target sequences can be nucleotide sequences (either DNA or RNA) or amino acid sequences. By way of example, a DNA target sequence for a three-finger ZFP is generally either 9 or 10 nucleotides in length, depending upon the presence and/or nature of cross-strand interactions between the ZFP and the target sequence.

The term “heterologous” is a relative term, which when used with reference to portions of a nucleic acid indicates that the nucleic acid comprises two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include an non-native (non-naturally occurring) nucleic acid that has integrated into the chromosome, or a non-native (non-naturally occurring) extrachromosomal nucleic acid.

Similarly, a heterologous protein indicates that the protein comprises two or more subsequences that are not found in the same relationship to each other in nature (e.g., a “fusion protein,” where the two subsequences are encoded by a single nucleic acid sequence). See, e.g., Ausubel, supra, for an introduction to recombinant techniques.

The term “recombinant,” when used with reference to a cell, indicates that the cell replicates an exogenous nucleic acid, or expresses a peptide or protein encoded by an exogenous nucleic acid. Recombinant cells can contain genes that are not found within the native (non-recombinant) form of the cell. Recombinant cells can also contain genes found in the native form of the cell wherein the genes are modified and re-introduced into the cell. A recombinant cell can comprise an unmodified cellular gene that has been introduced into the cell for the purpose, e.g., of overexpression. Expression of such an unmodified gene may be under the control of its normal cellular regulatory sequences or heterologous regulatory sequences. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette,” “expression cassette” or “expression construct” is a nucleic acid construct, generated recombinantly or synthetically, that has control elements that are capable of effecting expression of a structural gene that is operatively linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired polypeptide) and a promoter. Additional factors necessary or helpful in effecting expression can also be used as described herein. For example, an expression cassette can also include nucleotide sequences that encode a signal sequence that directs secretion of an expressed protein from the host cell, nuclear localization signals and/or epitope tags. Transcription termination signals, enhancers, and other nucleic acid sequences that influence gene expression, can also be included in an expression cassette.

“Kd” refers to the dissociation constant for a compound, i.e., the concentration of a compound (e.g., a zinc finger protein) that gives half maximal binding of the compound to its target (i.e., half of the compound molecules are bound to the target) under given conditions (i.e., when [target]<<Kd), as measured using a given assay system (see, e.g., U.S. Pat. No. 5,789,538). The assay system used to measure the Kd should be chosen so that it gives the most accurate measure of the actual Kd of the ZFP. Any assay system can be used, as long is it gives an accurate measurement of the actual Kd of the ZFP.

A “small molecule,” as disclosed herein, is a non-protein based moiety including, but not limited to the following: (i) molecules typically less than 10 K molecular weight; (ii) molecules that are permeable to cells, (iii) molecules that are less susceptible to degradation by many cellular mechanisms than peptides or oligonucleotides; and/or (iv) molecules that generally do not elicit an immune response. Many pharmaceutical companies have extensive libraries of chemical and/or biological mixtures, often fungal, bacterial, or algal extracts, or made by combinatorial chemistry techniques, that would be desirable to screen with the disclosed assays. Small molecules may be either biological or synthetic organic compounds, or even inorganic compounds (i.e., cisplatin).

A “hormone receptor” is a protein with hormone-dependent transcriptional regulatory activity. The nature of the regulatory activity of a hormone receptor depends upon whether or not the receptor is bound to its hormonal ligand. Hormone receptors can be nuclear or cytoplasmic. The nuclear hormone receptor (NHR) superfamily, members of which are often referred to as “nuclear receptors,” includes both nuclear and cytoplasmic hormone receptors.

Nuclear hormone receptors, when not bound to their ligand, are often able to bind to target DNA sequences, known as “response elements,” and generally repress transcription of the gene associated with the response element. In the presence of ligand, a DNA-bound nuclear receptor undergoes a conformational change that allows it to recruit coactivators, thereby activating transcription of its target gene.

Cytoplasmic hormone receptors, when unbound by their ligand, are localized in the cytoplasm of a cell through their association with chaperone proteins. Upon passage of the ligand across the cell membrane, binding of the ligand to the cytoplasmic receptor induces a conformational change that results in dissociation of the receptor from the chaperone protein. Release from the chaperone allows translocation of the receptor into the nucleus, where it bind response element sequences and modulates transcription of genes associated with the response element.

An “orphan receptor” is a hormone receptor whose ligand has not been identified.

Hormone receptors possess a DNA-binding domain, which is responsible for specific binding of the receptor to its cognate response element sequence. Hormone receptors also possess a ligand-binding domain, which is the portion of the molecule to which hormone binds and, in so doing, modulates the transcriptional regulatory function of the receptor.

“Therapeutic index” is a measure of how selective a drug is in producing its desired effects. It is often expressed as a ratio between the median lethal dose (LD₅₀) and the median effective dose (ED₅₀). In general, the higher the therapeutic index, the more likely that a drug will produce a desired effect in the absence of undesired side effects.

ZFP-Functional Domain Fusions for Multiplex Assays

Disclosed herein are compositions and methods for carrying out multiplex screening assays, which allow the simultaneous screening of multiple functional domains in a single cell population. The activity of each functional domain is assayed by measuring expression of a reporter gene that provides a readout specific to that functional domain. Correspondence between a first functional domain and a first reporter gene is created by constructing a fusion between the first functional domain and a zinc finger protein binding domain that is targeted to the first reporter gene. In like fashion, fusions between a second functional domain and a zinc finger protein binding domain targeted to a second reporter gene; and third, fourth, fifth, etc. functional domains fused to zinc finger protein binding domains targeted to third, fourth, fifth, etc. reporter genes can be constructed. All of the functional domains can be assayed simultaneously, since the products of the reporter genes can be easily distinguished, e.g., by RNA or protein analysis. In certain embodiments, a reporter gene is an endogenous cellular gene.

In certain embodiments, a plurality of drug targets (e.g., functional domains) are tested simultaneously. In additional embodiments, one of the functional domains is a drug target, and one or more additional functional domains is a related molecule (to test, e.g., for specificity), and/or an unrelated molecule and/or is involved in drug metabolism and/or is involved in drug toxicity. Each different functional domain is fused to a specific zinc finger protein (ZFP) binding domain and each ZFP binding domain is targeted to a different cellular reporter gene. Consequently, the effect of a drug on each of the functional domains can be determined by assaying expression of the reporter gene to which that functional domain is targeted by its attendant ZFP binding domain. In certain embodiments, a drug target is a nuclear hormone receptor.

Additional targets which can be simultaneously assayed by multiplexing, e.g., to test for specificity of a compound, include related protein family members, different protein isotypes, mutant protein isoforms, or proteins which are related to one another as RNA-splice variants. For example, it is possible to simultaneously assay related and/or unrelated proteins involved in similar or different signal transduction pathways. This type of analysis provides information on the specific ability of a test compound to regulate one or more particular protein drug targets. Increased drug specificity, obtained according to the practice of the present disclosure, will greatly reduce the amount of undesired side effects and will reduce the amount of time and cost that is currently required to study and optimize potential drug compounds in secondary screening assays.

Types of factors suitable for multiplexing can include related protein family members, different protein isotypes, mutant isoforms, or alternative RNA-splice variants. Other factors may include related or unrelated proteins involved in similar or different signal transduction pathways. Multiplexing with factors involved in the recognition, catabolic breakdown, and/or removal of foreign or toxic compounds (Xenobiotic receptors) would provide preliminary information on drug toxicology and metabolism, aiding in the identification compounds that are more potent, specific, and safe.

In certain embodiments, the same functional domain is targeted to a plurality of cellular reporter genes, to test for specificity of a drug. If expression of all of the reporter genes is modulated in a similar fashion, the specificity of the drug for the target is supported. A difference in the modulation of expression of the reporter genes suggests that the drug may modulate expression of one or more of the reporter gene independently of its molecular target.

The assay systems disclosed herein employ engineered ZFP technology by linking a desired signal transduction pathway to the expression of an endogenous cellular gene. This is achieved by fusing a peptide or functional domain(s) from a protein factor involved in transducing signals from extracellular ligands or stimuli to an engineered zinc finger protein (ZFP) DNA-binding domain targeted to an endogenous gene, creating a chimeric transcription factor that regulates the expression of the endogenous gene. This endogenous gene thus behaves as a reporter for the activity of the specific pathway of interest, and changes in the level of endogenous gene expression reflect the capacity of compounds to regulate the activity of specific protein targets, signal transduction pathways, and/or biological processes of interest. Gene expression can be monitored by methods that include RNA detection, e.g., TaqMan®, branched DNA (Quantigene, Bayer Corp.), eTags (Aclara), or microarrays (High Throughput Genomics); protein detection (e.g., ELISA-based assays, Luminex); or by biochemical or enzymatic assays (e.g., alkaline phosphatase assays).

The approach described in the preceding paragraphs can be multiplexed within a single cell line to increase screening throughput, create a method to decrease false positives, and to provide a small molecule screening platform that yields high information content on compound efficacy, specificity and toxicity/drug metabolism in a single assay system. Multiplexing is achieved by generating cell lines that simultaneously express different ZFPs fused to functional domains from related or unrelated signal transduction factors and/or nuclear receptors. Each engineered fusion molecule is targeted to a different endogenous reporter gene. Therefore, the ability of a compound to regulate one or more protein targets or biological processes can be determined by monitoring, simultaneously, changes in the expression of multiple reporter genes.

Since this screening platform employs endogenous genes as reporters, there is no theoretical limit to the number of reporter genes that can be used, or assays that can be multiplexed. By contrast, with existing reporter genes such as fluorescent proteins (e.g., GFP), the current limit of detection is three different types of fluorescent protein in a single cell. Similarly, the use of heterologous DNA-binding domains such as Gal4 or LexA is limited by the scarcity of well-characterized binding domain-target sequence pairs. Use of the present methods and compositions does not rely on previously characterized binding proteins and their target sites, because it is possible to design ZFP to bind virtually any sequence (see below).

An additional advantage of the disclosed multiplex assays is that fusion of the functional domain portion of the target protein to an engineered ZFP domain alters the DNA-binding characteristic of the target protein; thus, related factors with DNA-binding specificities similar to that of the target protein will not interfere with the assay by participating in regulation of the reporter gene. This type of interference is especially problematic with members of the nuclear hormone receptor superfamily, since many of these receptors share similar or identical DNA-binding characteristics.

Re-programming the DNA-binding specificity of a target protein, as disclosed herein, allows the simultaneous analyses of several targets in response to a compound, regardless of overlapping DNA-binding characteristics of, or endogenous genes regulated by, the native target molecules. Altering DNA-binding specificity also potentiates the isolation of more specific drugs that selectively regulate certain isotypes, mutant isoforms, or splice-variants of a drug target of interest.

Hormone Receptors

An exemplary functional domain is obtained from a hormone receptor, e.g., a nuclear receptor ligand-binding domain (LBD). Binding of a ligand to a nuclear receptor enables it to bind to DNA sequences termed “response elements.” Binding of a liganded nuclear receptor to its cognate response element can result in modulation of gene expression, e.g. by recruitment of co-activator or co-repressor complexes.

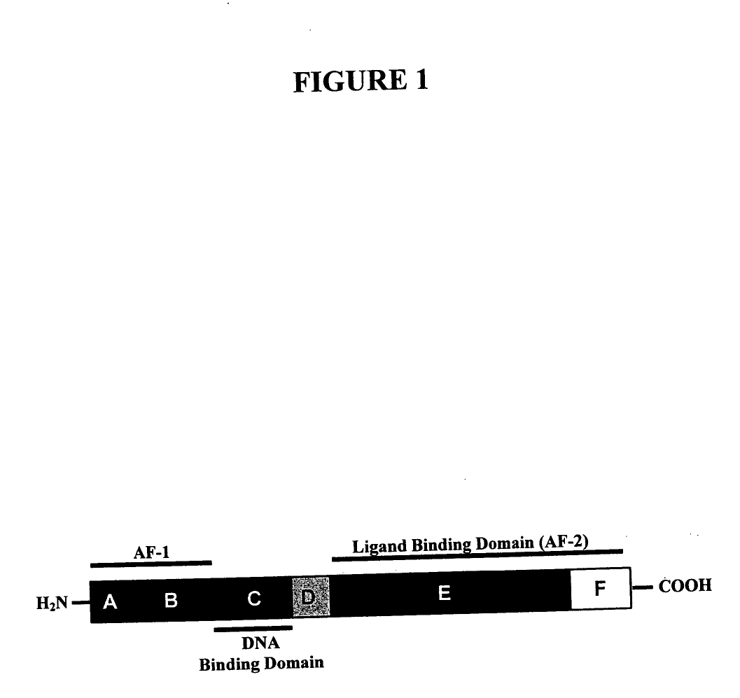

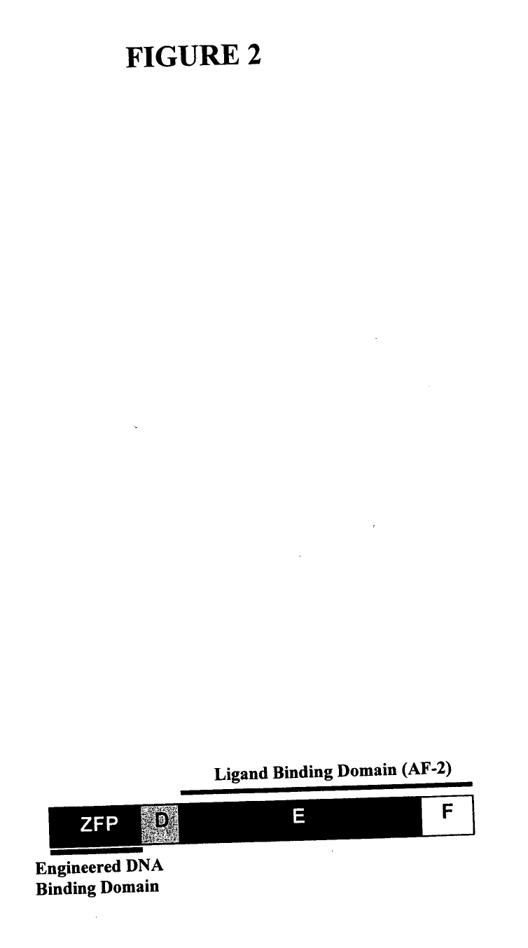

Nuclear receptors generally comprise separate ligand-binding and DNA-binding domains. See FIG. 1. The DNA-binding domain binds to hormone response element sequences in or near those genes that are normally regulated by the receptor. The inventors have discovered that the DNA-binding domain of a nuclear receptor can be replaced by an engineered zinc finger protein (ZFP) binding domain (see FIG. 2), thereby redirecting the biological activity of the nuclear receptor to one or more cellular genes not normally targeted by the receptor, which thereby become reporters for the activity of the receptor. Furthermore, the inventors have discovered that a plurality of LBD-ZFP fusions, each targeted to a different cellular reporter gene, can be simultaneously expressed in a cell under conditions in which each LBD-ZFP fusion is regulated by the ligand that normally regulates the receptor from which the LBD is derived. Thus, regulation of a cellular reporter gene, which is not normally regulated by the receptor, can be used as a readout for the activity of the receptor.

Exemplary nuclear receptors which can be screened in the multiplex assays disclosed herein include estrogen receptors (ERs), progesterone receptors (PRs), androgen receptors (ARs), glucocorticoid receptors (GRs), peroxisome proliferator-activated receptors (PPARs), retinoic acid receptors (RARs), retinoid X receptors (RXRs), vitamin D receptors, famesoid receptors (e.g., FXR), thyroid hormone receptors (TRs), androstane receptors (e.g., CARα, constitutive androstane receptor, MB67), liver receptors (e.g., LXR, liver X receptor), pregnane receptors (e.g., PXR, pregnane X receptor), SHP, HNF4A, MINOR, SF-1, COUP-TF, LRH-1 (NR5A2), TR3/Nurr77, DAX-1, and RORs, as well as various orphan receptors. In fact, the disclosed methods and compositions allow the rapid identification of ligands for orphan receptors, along with associated information on their specificity and toxicity, if desired.

Additional nuclear receptors are known to those of skill in the art. See, for example, Weatherman et al. (1999) Ann. Rev. Biochem. 68:559-581 and Aranda et al. (2001) Physiol. Rev. 81(3):1269-1304. See also U.S. Pat. Nos. 5,312,732; 5,571,696; 5,686,574; 5,696,233; 5,710,017; 5,756,448; 5,849,477; 5,958,710; 6,005,086; 6,222,015 and WO 96/21457; WO 96/22390; and WO 99/35246.

Zinc Finger Protein Binding Domains

As disclosed herein, multiplex assays employ a plurality of fusion molecules, wherein each fusion molecule comprises a fusion between a functional domain and a zinc finger DNA-binding domain. Zinc finger DNA-binding domains are described, for example, in Miller et al. (1985) EMBO J. 4:1609-1614; Rhodes et al. (1993) Scientific American Feb.:56-65; and Klug (1999) J. Mol. Biol. 293:215-218. The three-fingered Zif268 murine transcription factor has been particularly well studied. Pavletich, N. P. & Pabo, C. O. (1991) Science 252:809-1). The X-ray co-crystal structure of Zif268 ZFP and its double-stranded DNA target sequence indicates that each finger interacts independently with DNA. Nolte et al. (1998) Proc Natl Acad Sci USA 95:2938-2943; Pavletich, N. P. & Pabo, C. O. (1993) Science 261:1701-1707. The organization of the 3-fingered domain allows recognition of three to four contiguous base-pair triplets by each finger. Each finger is approximately 30 amino acids long, adopting a ββα fold. The two β-strands form a sheet, positioning the recognition α-helix in the major groove for DNA binding. Specific contacts with the bases are mediated primarily by four amino acids immediately preceding and within the recognition helix. Conventionally, these recognition residues are numbered −1, 2, 3, and 6 based on their positions in the α-helix.

ZFP DNA-binding domains are engineered (e.g., designed and/or selected) to recognize a particular target site as described in U.S. Pat. Nos. 5,789,538; 6,007,408; 6,013,453; 6,140,081; 6,140,466; 6,242,568 and 6,453,242; and PCT publications WO 95/19431, WO 98/53057, WO 98/53058, WO 98/53059, WO 98/53060, WO 98/54311, WO 00/23464, WO 00/27878, WO 00/41566, WO 00/42219, WO 01/53480 and WO 02/42459. In one embodiment, a target site for a zinc finger DNA-binding domain is identified according to site selection rules disclosed in co-owned U.S. Pat. No. 6,453,242. In certain embodiments, a ZFP is selected by iterative processes of selection and optimization as described in co-owned International Patent Application PCT/JUS01/43568. In additional embodiments, the binding specificity of the DNA-binding domain can be determined by identifying accessible regions in the sequence in question (e.g., in cellular chromatin). Accessible regions can be determined as described in co-owned PCT publications WO 01/83732 and WO 01/83751, the disclosures of which are hereby incorporated by reference herein. A DNA-binding domain is then designed and/or selected as described herein to bind to a target site within the accessible region.

Two alternative methods are typically used to create the coding sequences required to express newly designed DNA-binding peptides. One protocol is a PCR-based assembly procedure that utilizes six overlapping oligonucleotides. Three oligonucleotides correspond to “universal” sequences that encode portions of the DNA-binding domain between the recognition helices. These oligonucleotides remain constant for all zinc finger constructs. The other three “specific” oligonucleotides are designed to encode the recognition helices. These oligonucleotides contain substitutions primarily at positions −1, 2, 3 and 6 on the recognition helices making them specific for each of the different DNA-binding domains.

The PCR synthesis is carried out in two steps. First, a double stranded DNA template is created by combining the six oligonucleotides (three universal, three specific) in a four cycle PCR reaction with a low temperature annealing step, thereby annealing the oligonucleotides to form a DNA “scaffold.” The gaps in the scaffold are filled in by high-fidelity thermostable polymerase, the combination of Taq and Pfu polymerases also suffices. In the second phase of construction, the zinc finger template is amplified by external primers designed to incorporate restriction sites at either end for cloning into a shuttle vector or directly into an expression vector.

An alternative method of cloning the newly designed DNA-binding proteins relies on annealing complementary oligonucleotides encoding the specific regions of the desired zinc finger protein. This particular application requires that the oligonucleotides be phosphorylated prior to the final ligation step. Phosphorylation is usually performed before annealing, but can also be done post-annealing. In brief, the “universal” oligonucleotides encoding the constant regions of the proteins are annealed with their complementary oligonucleotides. Additionally, the “specific” oligonucleotides encoding the finger recognition helices are annealed with their respective complementary oligonucleotides. These complementary oligos are designed to fill in the region, which was previously filled in by polymerase in the protocol described above. The complementary oligos to the common oligos 1 and finger 3 are engineered to leave overhanging sequences specific for the restriction sites used in cloning into the vector of choice. The second assembly protocol differs from the initial protocol in the following aspects: the “scaffold” encoding the newly designed zinc finger protein is composed entirely of synthetic DNA thereby eliminating the polymerase fill-in step, additionally the fragment to be cloned into the vector does not require amplification. Lastly, inclusion in the design of sequence-specific overhangs eliminates the need for restriction enzyme digestion of the ZFP-encoding fragment prior to its insertion into the vector.

The resulting fragment encoding the newly designed zinc finger protein is ligated into an expression vector. Expression vectors that are commonly utilized include, but are not limited to, a modified pMAL-c2 bacterial expression vector (New England BioLabs, “NEB”) or a eukaryotic expression vector, pcDNA (Promega). Conventional methods of purification can be used (see Ausubel, supra, Sambrook, supra). In addition, any suitable host can be used, e.g., bacterial cells, insect cells, yeast cells, mammalian cells, and the like.

Expression of the zinc finger protein fused to a maltose binding protein (MBP-ZFP) in bacterial strain JM109 allows for straightforward purification through an amylose column (NEB). High expression levels of the zinc finger chimeric protein can be obtained by induction with IPTG since the MBP-ZFP fusion in the pMal-c2 expression plasmid is under the control of the IPTG inducible tac promoter (NEB). Bacteria containing the MBP-ZFP fusion plasmids are inoculated in to 2×YT medium containing 10 μM ZnCl₂, 0.02% glucose, plus 50 μg/ml ampicillin and shaken at 37° C. At mid-exponential growth IPTG is added to 0.3 mM and the cultures are allowed to shake. After 3 hours the bacteria are harvested by centrifugation, disrupted by sonication, and then insoluble material is removed by centrifugation. The MBP-ZFP proteins are captured on an amylose-bound resin, washed extensively with buffer containing 20 mM Tris-HCl (pH 7.5), 200 mM NaCl, 5 mM DTT and 50 μM ZnCl₂, then eluted with maltose in essentially the same buffer (purification is based on a standard protocol from NEB). Purified proteins are quantitated and stored for biochemical analysis.

The biochemical properties of the purified proteins, e.g., K_{d}, can be characterized by any suitable assay. K_{d} can be characterized via electrophoretic mobility shift assays (“EMSA”) (Buratowski & Chodosh, in Current Protocols in Molecular Biology pp. 12.2.1-12.2.7 (Ausubel ed., 1996); see also U.S. Pat. No. 5,789,538, and PCT WO 00/42219, herein incorporated by reference). Affinity is measured by titrating purified protein against a low fixed amount of labeled double-stranded oligonucleotide target. The target comprises the natural binding site sequence (e.g., 9 or 18 bp), optionally flanked by the 3 bp found in the natural sequence. External to the binding site plus flanking sequence is a constant sequence. The annealed oligonucleotide targets possess a 1-nucleotide 5′ overhang that allows for efficient labeling of the target with T4 phage polynucleotide kinase. For the assay the target is added at a concentration of 40 nM or lower (the actual concentration is kept at least 10-fold lower than the lowest protein dilution) and the reaction is allowed to equilibrate for at least 45 min. In addition the reaction mixture also contains 10 mM Tris (pH 7.5), 100 mM KCl, 1 mM MgCl₂, 0.1 mM ZnCl₂, 5 mM DTT, 10% glycerol, 0.02% BSA (poly (dIdC) or (dAdT) (Pharmacia) can also added at 10-100 μg/μl).

The equilibrated reactions are loaded onto a 10% polyacrylamide gel, which has been pre-run for 45 min in Tris/glycine buffer, then bound and unbound labeled target is resolved be electrophoresis at 150V (alternatively, 10-20% gradient Tris-HCl gels, containing a 4% polyacrylamide stacker, can be used). The dried gels are visualized by autoradiography or phosphoroimaging and the apparent K_{d} is determined by calculating the protein concentration that gives half-maximal binding.

Similar assays can also include determining active fractions in the protein preparations. Active fractions are determined by stoichiometric gel shifts where proteins are titrated against a high concentration of target DNA. Titrations are done at 100, 50, and 25% of target (usually at micromolar levels).

Fusion Molecules

In the compositions and methods described herein, zinc finger-containing proteins that target specific sequences are generally provided as fusion molecules in combination with other molecules, particularly with one or more functional domains. Thus, in certain embodiments, the compositions and methods disclosed herein involve one or more fusions between a zinc finger protein (or functional fragments thereof) and one or more functional domains such as, for example, a nuclear hormone receptor ligand binding domain (or functional fragment thereof), or a polynucleotide encoding such a fusion. Changes in regulation of multiple distinct target gene by a plurality of fusion proteins provides a multiplex assay for drug screening, as disclosed herein.

The zinc finger protein can be covalently or non-covalently associated with one or more functional domains, alternatively two or more functional domains, with the two or more domains being two copies of the same domain, or two different domains. The functional domains can be covalently linked to the zinc finger protein, e.g., via an amino acid linker, as part of a fusion protein. The zinc finger proteins can also be associated with a functional domain via a non-covalent dimerization domain, e.g., a leucine zipper, a STAT protein N terminal domain, or a protein that binds cyclosporine, tetracycline, a steroid, FK506, FK520, rapamycin, and analogues or derivatives thereof. Examples of such proteins include FK506 binding proteins (FKBPs), cyclophilin receptors, tetracycline receptors, steroid receptors and FRAPs. See, e.g., U.S. Pat. No. 6,165,787; O'Shea, Science 254: 539 (1991), Barahmand-Pour et al., Curr. Top. Microbiol. Immunol. 211:121-128 (1996); Klemm et al., Annu. Rev. Immunol. 16:569-592 (1998); Ho et al., Nature 382:822-826 (1996); and Pomeranz et al., Biochem. 37:965 (1998). The regulatory domain can be associated with the zinc finger protein at any suitable position, including the C- or N-terminus of the zinc finger protein.

Fusion molecules can be constructed by methods of cloning and biochemical conjugation that are well known to those of skill in the art. In certain embodiments, fusion molecules comprise a zinc finger protein and one or more functional domains. Optionally, fusion molecules also comprise nuclear localization signals (such as, for example, that from an SV40 T-antigen) and epitope tags (such as, for example, FLAG, myc and hemagglutinin). Fusion proteins (and nucleic acids encoding them) are designed such that the translational reading frame is preserved among the components of the fusion.

Linker domains between polypeptide domains, e.g., between the zinc finger proteins and a functional domain, can be included. Such linkers are typically polypeptide sequences, such as poly gly sequences of between about 5 and 200 amino acids. Preferred linkers are typically flexible amino acid subsequences that are synthesized as part of a recombinant fusion protein, for example, the linkers DGGGS (SEQ ID NO: 1); TGEKP (SEQ ID NO: 2) (see, e.g., Liu et al., Proc. Natl. Acad. Sci. U.S.A. 5525-5530 (1997)); LRQKDGERP (SEQ ID NO: 3); GGRR (SEQ ID NO: 4) (Pomerantz et al. 1995, supra); (G₄S)ₙ (SEQ ID NO: 5) (Kim et al., Proc. Natl. Acad. Sci. U.S.A. 93, 1156-1160 (1996); GGRRGGGS (SEQ ID NO: 6); LRQRDGERP (SEQ ID NO: 7); LRQKDGGGSERP (SEQ ID NO: 8); and LRQKd(G₃S)₂ERP (SEQ ID NO: 9). Additional suitable linkers are disclosed in WO 99/45132 and WO 01/53480.

A chemical linker can be used to connect synthetically or recombinantly produced domain sequences. For example, poly(ethylene glycol) linkers are available from Shearwater Polymers, Inc. Huntsville, Ala. Some linkers have amide linkages, sulfhydryl linkages, or heterofunctional linkages. In addition to covalent linkage of zinc finger proteins to regulatory domains, non-covalent methods can be used to produce molecules with zinc finger proteins associated with regulatory domains. See, for example, U.S. Pat. No. 6,165,787 and WO 01/30843.

As noted above, the fusion molecules may be in the form of nucleic acid sequences that encode the fusion molecule, or in the form of a fusion between one or more polypeptides and/or one or more polypeptides and one or more non-polypeptide molecules.

Reporter Genes

The fusion molecules disclosed herein comprise a zinc finger binding protein that binds to a target site (in a reporter gene) and functional domain. Preferably, the target site is in an endogenous gene whose level of expression can be readily assayed. Modulation of gene expression can be in the form of increased expression or repression. The effect of a compound or substance on the regulation of the reporter gene by the fusion protein can then be determined as part of a multiplex screening assay.

Any cellular gene, whose product can be detected, can be used as a reporter gene. Detection of a gene product can include, for example, detection of RNA, detection of protein, or detection of enzymatic activity of a protein gene product (e.g., phosphatase, peroxidase, galactosidase, glucuronidase). Preferred are genes whose products can be assayed in high-throughput fashion by e.g., ELISA, enzymatic assays or RNA detection. Exemplary reporter genes include, but are not limited to, cyclin-dependent kinase inhibitor p57 (kip2), gastrin-releasing peptide (GRP), annexins (e.g., AnxA8), insulin-like growth factors (IGFs), alkaline phosphatses, keratins, e.g., keratin 5 (krt5) and cystatin SN.

Virtually any component of a cell can serve as a molecular target (reporter) for the ZFP component of the fusion protein. For example, the product (mRNA or protein) of an endogenous cellular genes such as, e.g., VEGF, H19 or IGF-2, can serve as reporter. A gene whose product is used as a reporter is denoted a “reporter gene.” An exogenous gene can also serve as a reporter gene, for example, if it is integrated into the chromosome so that it adopts a chromatin configuration. Additional non-limiting examples of endogenous reporters include growth factor receptors (e.g., FGFR, PDGFR, EGFR, NGFR, and VEGFR). Other endogenous reporters are G-protein receptors and include substance K receptor, the angiotensin receptor, the α- and β-adrenergic receptors, the serotonin receptors, and PAF receptor. See, e.g., Gilman, Ann. Rev. Biochem. 56:625-649 (1987). Other suitable reporters that may be employed include ion channels (e.g., calcium, sodium, potassium channels), muscarinic receptors, acetylcholine receptors, GABA receptors, glutamate receptors, and dopamine receptors (see Harpold, 5,401,629 and U.S. Pat. No. 5,436,128). Other targets are adhesion proteins such as integrins, selectins, and immunoglobulin superfamily members (see Springer, Nature 346:425-433 (1990). Osborn (199) Cell 62:3; Hynes (1992) Cell 69:11). Other endogenous reporters are cytokines, such as interleukins IL-1 through IL-13, tumor necrosis factors α & β, interferons α, β and γ, transforming growth factor Beta (TGF-β), colony stimulating factor (CSF) and granulocyte-macrophage colony stimulating factor (GM-CSF). See Human Cytokines: Handbook for Basic & Clinical Research (Aggrawal et al. eds., Blackwell Scientific, Boston, Mass. 1991). Target molecules that serve as reporter molecules can be human, mammalian viral, plant, fungal or bacterial. Other targets are antigens, such as proteins, glycoproteins and carbohydrates from microbial pathogens, both viral and bacterial, and tumors. Still other targets are described in U.S. Pat. No. 4,366,241.

Additional examples of target genes suitable for use as reporters include VEGF, CCR5, ERα, Her2/Neu, Tat, Rev, HBV C, S, X, and P, LDL-R, PEPCK, CYP7, Fibrinogen, ApoB, Apo E, Apo(a), renin, NF-κB, I-κB, TNF-α, FAS ligand, amyloid precursor protein, atrial naturetic factor, ob-leptin, ucp-1, IL-1, IL-2, IL-3, IL-4, IL-5, IL-6, IL-12, G-CSF, GM-CSF, Epo, PDGF, PAF, p53, Rb, fetal hemoglobin, dystrophin, eutrophin, GDNF, NGF, IGF-1, VEGF receptors flt and flk, topoisomerase, telomerase, bcl-2, cyclins, angiostatin, IGF, ICAM-1, STATS, c-myc, c-myb, TH, PTI-1, polygalacturonase, EPSP synthase, FAD2-1, delta-12 desaturase, delta-9 desaturase, delta-15 desaturase, acetyl-CoA carboxylase, acyl-ACP-thioesterase, ADP-glucose pyrophosphorylase, starch synthase, cellulose synthase, sucrose synthase, senescence-associated genes, heavy metal chelators, fatty acid hydroperoxide lyase, viral genes, protozoal genes, fungal genes, and bacterial genes. In general, suitable reporter genes include cytokines, lymphokines, growth factors, mitogenic factors, chemotactic factors, onco-active factors, receptors, potassium channels, G-proteins, signal transduction molecules, and other disease-related genes.

Modulation of reporter gene expression can be assayed by determining any parameter that is indirectly or directly affected by the expression of the target gene. Such parameters include, e.g., changes in RNA or protein levels, changes in protein activity, changes in product levels, changes in downstream gene expression, changes in signal transduction, phosphorylation and dephosphorylation, receptor-ligand interactions, second messenger concentrations (e.g., cGMP, cAMP, IP3, and Ca²⁺), cell growth, and neovascularization, etc., as described herein. These assays can be in vitro, in vivo, and ex vivo. Such functional effects can be measured by any means known to those skilled in the art, e.g., measurement of RNA or protein levels, measurement of RNA stability, identification of downstream or reporter gene expression, e.g., via chemiluminescence, fluorescence, calorimetric reactions, antibody binding, inducible markers, ligand binding assays; changes in intracellular second messengers such as cGMP and inositol triphosphate (IP3); changes in intracellular calcium levels; cytokine release, and the like, as described herein.

Reporter expression can be directly detected by detecting formation of transcript or of translation product. For example, transcription product can be detected using Northern blots, branched DNA signal amplification systems (e.g., U.S. Pat. Nos. 5,124,246; 5,624,802; 5,635,352; 5,681,697; 5,849,481), RNA tags (Aclara Biosciences, Mountain View, Calif.) or real-time PCR (Taqman®, Roche) and the formation of certain proteins can be detected, e.g., by gel electrophoresis, immunoassay (e.g., ELISA), using a characteristic stain or by detecting an inherent characteristic (e.g., enzymatic activity) of the protein. Additionally, expression of reporter can be determined by detecting a product formed as a consequence of an activity of the reporter.

Exemplary reporter genes encoding proteins having enzymatic activity include, but are not limited to, those encoding phosphatases, hydrolases, myeloperoxidases and proteases. Additional exemplary reporter genes include those encoding cell-surface proteins such as, for example, CD antigens, immunoglobulins, T-cell receptors, growth factor receptors and transmembrane proteins (e.g., placental alkaline phosphatase).

Other reporters are enzymes that catalyze the formation of a detectable product. Suitable enzymes include proteases, nucleases, liposes, phosphatases, sugar hydrolases and esterases. Preferably, the substrate is substantially impermeable to eukaryotic plasma membranes, thus making it possible to tightly control signal formation. Examples of suitable reporter genes that encode enzymes include, for example, CAT (chloramphenicol acetyl transferase; Alton and Vapnek (1979) Nature 282:864-869), luciferase (lux), β-galactosidase, β-glucuronidase (GUS) and alkaline phosphatase (Toh, et al. (1980) Eur. J. Biochem. 182:231-238; and Hall et al. (1983) J. Mol. Appl. Gen. 2:101).

In addition to, or instead of, assessing mRNA or protein expression, a variety of different cellular and/or biochemical responses (also termed cell properties) can also be measured and compared in the methods described herein. For example, the cellular response to administration of a compound can be quantified as a value or level of a cellular property, such as cell growth, neovascularization, hormone release, pH changes, changes in intracellular second messengers such as GMP, receptor binding and the like. The units of the value depend on the property. For example, the units can be units of absorbance, photon count, radioactive particle count or optical density.

Functional Domains

The fusion molecules disclosed herein include one or more regulatory (functional) domains including, e.g., effector domains from transcription factors (activators, repressors, co-activators, co-repressors), silencers, nuclear hormone receptors, oncogene transcription factors (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases, deacetylases, phosphatases, methyltransferases, ubiquitinylases); and DNA modifying enzymes (e.g., methyltransferases, topoisomerases, helicases, ligases, kinases, phosphatases, polymerases, and/or endonucleases, and their associated factors and modifiers.

Transcription factor polypeptides from which regulatory domains can be obtained include those that are involved in regulated and basal transcription. Such polypeptides include transcription factors, their effector domains, coactivators, silencers, nuclear hormone receptors (see, e.g., Goodrich et al., Cell 84:825-30 (1996) for a review of proteins and nucleic acid elements involved in transcription; transcription factors in general are reviewed in Barnes & Adcock, Clin. Exp. Allergy 25 Suppl. 2:46-9 (1995) and Roeder, Methods Enzymol. 273:165-71 (1996)). Databases dedicated to transcription factors are known (see, e.g., Science 269:630 (1995)). Nuclear hormone receptor transcription factors are described in, for example, Rosen et al., J. Med. Chem. 38:4855-74 (1995). The C/EBP family of transcription factors are reviewed in Wedel et al., Immunobiology 193:171-85 (1995). Coactivators and co-repressors that mediate transcription regulation by nuclear hormone receptors are reviewed in, for example, Meier, Eur. J. Endocrinol. 134(2):158-9 (1996); Kaiser et al., Trends Biochem. Sci. 21:342-5 (1996); and Utley et al., Nature 394:498-502 (1998)). GATA transcription factors, which are involved in regulation of hematopoiesis, are described in, for example, Simon, Nat. Genet. 11:9-11 (1995); Weiss et al., Exp. Hematol. 23:99-107. TATA box binding protein (TBP) and its associated TAF polypeptides (which include TAF30, TAF55, TAF80, TAF110, TAF150, and TAF250) are described in Goodrich & Tjian, Curr. Opin. Cell Biol. 6:403-9 (1994) and Hurley, Curr. Opin. Struct. Biol. 6:69-75 (1996). The STAT family of transcription factors are reviewed in, for example, Barahmand-Pour et al., Curr. Top. Microbiol. Immunol. 211:121-8 (1996). Transcription factors involved in disease are reviewed in Aso et al., J. Clin. Invest. 97:1561-9 (1996).

Additional functional domains are disclosed, for example, in co-owned WO 00/41566.

Useful domains can also be obtained from the gene products of oncogenes (e.g., myc, jun, fos, myb, max, mad, rel, ets, bcl, myb, mos family members) and their associated factors and modifiers. Oncogenes are described in, for example, Cooper, Oncogenes, The Jones and Bartlett Series in Biology (2^{nd} ed., 1995). The ets transcription factors are reviewed in Waslylk et al., Eur. J. Biochem. 211:7-18 (1993) and Crepieux et al., Crit. Rev. Oncog. 5:615-38 (1994). Myc oncogenes are reviewed in, for example, Ryan et al., Biochem. J. 314:713-21 (1996). The jun and fos transcription factors are described in, for example, The Fos and Jun Families of Transcription Factors (Angel & Herrlich, eds. 1994). The max oncogene is reviewed in Hurlin et al., Cold Spring Harb. Symp. Quant. Biol. 59:109-16. The myb gene family is reviewed in Kanei-Ishii et al., Curr. Top. Microbiol. Immunol. 211:89-98 (1996). The mos family is reviewed in Yew et al., Curr. Opin. Genet. Dev. 3:19-25 (1993).

In addition to functional domains, often the zinc finger protein is expressed as a fusion protein such as maltose binding protein (“MBP”), glutathione S transferase (GST), hexahistidine, c-myc, and the FLAG epitope, for ease of purification, monitoring expression, or monitoring cellular and subcellular localization.

Compounds

The methods and compositions described herein are useful in screening a wide variety of compounds. For example, compounds to be screened in the present multiplex assays can be obtained from combinatorial libraries of peptides or small molecules, can be hormones, growth factors, and cytokines, can be naturally occurring molecules or can be from existing repertoires of chemical compounds synthesized by the pharmaceutical industry. Combinatorial libraries can be produced for many types of compound that can be synthesized in a step-by-step fashion. Such compounds include polypeptides, beta-turn mimetics, polysaccharides, nucleic acids, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated by reference for all purposes). Peptide libraries can also be generated by phage display methods. See, e.g., Devlin, WO 91/18980. Compounds to be screened can also be obtained from the National Cancer Institute's Natural Product Repository, Bethesda, Md. Existing compounds or drugs with known efficacy can also be screened to evaluate side effects.

Delivery

When the molecular target is intracellular, a compound that interacts with it must traverse the cell membrane. The compound can be administered directly into a cell using methods known in the art and described herein. A compound contacted with a cell can cross the cell membrane in a number of ways. If the compound has suitable size and charge properties, it can be passively transported across the membrane. Other processes of membrane passage include active transport (e.g., receptor mediated transport), endocytosis and pinocytosis. Where a compound cannot be effectively transported by any of the preceding methods, microinjection, biolistics or other methods can be used to deliver it to the internal portion of the cell. Alternatively, if the compound to be screened is a protein, a nucleic acid encoding the protein can be introduced into the cell and expressed within the cell.

Likewise, the zinc finger protein-functional domain fusions for use in the multiplex assay must be introduced into the cell. Typically such is achieved either by introducing the ZFP-functional domain molecule into a cell or by introducing a nucleic acid encoding the ZFP-functional domain fusion into the cell, resulting in expression of the fusion protein within the cell. Nucleic acids can be introduced by conventional means including viral based methods, chemical methods, lipofection and microinjection. The introduced nucleic acid can integrate into the host chromosome, persist in episomal form or can have a transient existence in the cytoplasm. Similarly, an exogenous protein can be introduced into a cell in protein form. For example, the zinc finger protein can be introduced by lipofection, biolistics, microinjection or through fusion to membrane translocating domains.

Thus, the compositions described herein can be provided to the target cell in vitro or in vivo. In addition, the compositions can be provided as polypeptides, polynucleotides or combination thereof. In certain embodiments, the fusion molecule is constitutively expressed. In other embodiments, expression of the ZFP-functional domain fusion is controlled by an inducible promoter.

A. Delivery of Polynucleotides

In certain embodiments, the compositions are provided as one or more polynucleotides. Further, as noted above, a zinc finger protein-containing composition can be designed as a fusion between a polypeptide zinc finger and one or more functional domains (e.g., a ligand binding domain), that is encoded by a fusion nucleic acid. In both fusion and non-fusion cases, the nucleic acid can be cloned into intermediate vectors for transformation into prokaryotic or eukaryotic cells for replication and/or expression. Intermediate vectors for storage or manipulation of the nucleic acid or production of protein can be prokaryotic vectors, (e.g., plasmids), shuttle vectors, insect vectors, or viral vectors for example. A nucleic acid encoding a zinc finger protein can also cloned into an expression vector, for administration to a bacterial cell, fungal cell, protozoal cell, piscine cell, plant cell, or animal cell, preferably a mammalian cell, more preferably a human cell.

To obtain expression of a cloned nucleic acid, it is typically subcloned into an expression vector that contains a promoter to direct transcription. Suitable bacterial and eukaryotic promoters are well known in the art and described, e.g., in Sambrook et al., supra; Ausubel et al., supra; and Kriegler, Gene Transfer and Expression: A Laboratory Manual (1990). Bacterial expression systems are available in, e.g., E. coli, Bacillus sp., and Salmonella. Palva et al. (1983) Gene 22:229-235. Kits for such expression systems are commercially available. Eukaryotic expression systems for mammalian cells, yeast, and insect cells are well known in the art and are also commercially available, for example, from Invitrogen, Carlsbad, Calif. and Clontech, Palo Alto, Calif.

The promoter used to direct expression of the nucleic acid of choice depends on the particular application. For example, a strong constitutive promoter is typically used for expression and purification. In contrast, when a protein is to be used in vivo, either a constitutive or an inducible promoter is used, depending on the particular use of the protein. In addition, a weak promoter can be used, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system. See, e.g., Gossen et al. (1992) Proc. Natl. Acad. Sci USA 89:5547-5551; Oligino et al.(1998) Gene Ther. 5:491-496; Wang et al. (1997) Gene Ther. 4:432-441; Neering et al. (1996) Blood 88:1147-1155; and Rendahl et al. (1998) Nat. Biotechnol. 16:757-761.

In addition to a promoter, an expression vector typically contains a transcription unit or expression cassette that contains additional elements required for the expression of the nucleic acid in host cells, either prokaryotic or eukaryotic. A typical expression cassette thus contains a promoter operably linked, e.g., to the nucleic acid sequence, and signals required, e.g., for efficient polyadenylation of the transcript, transcriptional termination, ribosome binding, and/or translation termination. Additional elements of the cassette may include, e.g., enhancers, and heterologous spliced intronic signals.

A variety of inducible promoters (e.g., operably linked to control expression of a polynucleotide encoding a fusion protein) can be used, for example the tet-repressor system. Gossen et al. Science (1995) 268:1766-1769, describe fusion of a tetracycline resistance gene repressor to a viral transcription activation domain in order to induce rapid, greatly amplified gene expression in the presence of tetracycline. It is a modification of a preexisting system in which low levels of tetracycline prevented gene expression. The gene that codes for the tetracycline resistance gene repressor was mutagenized and a mutant fusion protein was created that depended on tetracycline for activation was identified. The construct can provide an on/off switch for high expression of a gene.

Other activator/promoter sequences known in the art may also be used in construction of plasmids for expression of fusion molecules. These include, but are not limited to: (1) the T7 lac promoter construct activated by T7 RNA polymerase as the transactivator (Dubendorfs & Studier, J. Mol. Biol., 219: 45-49, 1991); (2) the Lex A (binding domain)/Gal4 transcriptional activator-for the Lex A promoter (Brent & Ptashne, Cell 43: 729-736, 1985); (3) Gal4NVP16 (Carey et al., J- Mol. Biol. 209: 423-432, 1989; Cress et al., Science, 251: 87-90, 1991; Sadowski et al. Nature, 335: 563-564, 1988); (4) lac operator/repressor system as modified for eukaryotic expression (Brown et al., Cell 49: 603-612, 1987); (5) T7 polymerase-vaccinia virus promoter system (Fuerst et al., Proc. Natl. Acad. Sci. USA 83: 8122-8126; Fuerst et al., Molec. Cell Biol. 7: 2538-2544, 1987); (6) the T3 lac constructs activated by T3 RNA polymerase as the transactivator (Deuschle et al., Proc. Natl. Acad. Sci. USA 86: 5400-5404, 1989); and (7) glucocorticoid inducible mouse mammary tumor virus promoter system, (Lee et al., Nature 294: 228-232, 1981; Huang et al., Cell 27: 245-256, 1981; Ostrowski et al., Mol Cell. Biol. 3: 2045-2057, 1983). The tet operator/eCMV promoter exemplified herein also may be modified to comprise the vaccinia virus promoter (Fuerst et al., 1987, supra) instead of the eCMV promoter.

The particular expression vector used to transport the genetic information into the cell is selected with regard to the intended use of the resulting ZFP polypeptide, e.g., expression in plants, animals, bacteria, fungi, protozoa etc. Standard bacterial expression vectors include plasmids such as pBR322, pBR322-based plasmids, pSKF, pET23D, and commercially available fusion expression systems such as GST and LacZ. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High-yield expression systems are also suitable, such as baculovirus vectors in insect cells, with a nucleic acid sequence coding for a ZFP as described herein under the transcriptional control of the polyhedrin promoter or any other strong baculovirus promoter.

Elements that are typically included in expression vectors also include a replicon that functions in E. coli (or in the prokaryotic host, if other than E. coli), a selective marker, e.g., a gene encoding antibiotic resistance, to permit selection of bacteria that harbor recombinant plasmids, and unique restriction sites in nonessential regions of the vector to allow insertion of recombinant sequences.

Standard transfection methods can be used to produce bacterial, mammalian, yeast, insect, or other cell lines that express large quantities of zinc finger proteins, which can be purified, if desired, using standard techniques. See, e.g., Colley et al. (1989) J. Biol. Chem. 264:17619-17622; and Guide to Protein Purification, in Methods in Enzymology, vol. 182 (Deutscher, ed.) 1990. Transformation of eukaryotic and prokaryotic cells are performed according to standard techniques. See, e.g., Morrison (1977) J. Bacteriol. 132:349-351; Clark-Curtiss et al. (1983) in Methods in Enzymology 101:347-362 (Wu et al., eds).

Any procedure for introducing foreign nucleotide sequences into host cells can be used. These include, but are not limited to, the use of calcium phosphate transfection, DEAE-dextran-mediated transfection, polybrene, protoplast fusion, electroporation, lipid-mediated delivery (e.g., liposomes), microinjection, particle bombardment, introduction of naked DNA, plasmid vectors, viral vectors (both episomal and integrative) and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

Conventional viral and non-viral based nucleic acid delivery methods can be used to introduce nucleic acids into host cells or target tissues. Such methods can be used to administer nucleic acids encoding reprogramming polypeptides to cells in vitro. Additionally, nucleic acids are administered for in vivo or ex vivo. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For reviews of nucleic acid delivery procedures, see, for example, Anderson (1992) Science 256:808-813; Nabel et al. (1993) Trends Biotechnol. 11:211-217; Mitani et al. (1993) Trends Biotechnol. 11:162-166; Dillon (1993) Trends Biotechnol. 11:167-175; Miller (1992) Nature 357:455-460; Van Brunt (1988) Biotechnology 6(10):1149-1154; Vigne (1995) Restorative Neurology and Neuroscience 8:35-36; Kremer et al. (1995) British Medical Bulletin 51(1):31-44; Haddada et al., in Current Topics in Microbiology and Immunology, Doerfler and Böhm (eds), 1995; and Yu et al. (1994) Gene Therapy 1:13-26.

Methods of non-viral delivery of nucleic acids include lipofection, microinjection, ballistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386; 4,946,787; and 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424 and WO 91/16024. Nucleic acid can be delivered to cells (ex vivo administration) or to target tissues (in vivo administration).

The preparation of lipid:nucleic acid complexes, including targeted liposomes such as immunolipid complexes, is well known to those of skill in the art. See, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; and U.S. Pat. Nos. 4,186,183; 4,217,344; 4,235,871; 4,261,975; 4,485,054; 4,501,728; 4,774,085; 4,837,028 and 4,946,787.

The use of RNA or DNA virus-based systems for the delivery of nucleic acids take advantage of highly evolved processes for targeting a virus to specific cells in the body and trafficking the viral payload to the nucleus. Viral vectors can be administered directly to subjects (in vivo) or they can be used to treat cells in vitro, wherein the modified cells are administered to subjects (ex vivo). Conventional viral based systems for the delivery of ZFPs include retroviral, lentiviral, poxviral, adenoviral, adeno-associated viral, vesicular stomatitis viral and herpes viral vectors. Integration in the host genome is possible with certain viral vectors, including the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, allowing alteration and/or expansion of the potential target cell population. Lentiviral vectors are retroviral vector that are able to transduce or infect non-dividing cells and typically produce high viral titers. Selection of a retroviral nucleic acid delivery system would therefore depend on the target cell and/or tissue. Retroviral vectors have a packaging capacity of up to 6-10 kb of foreign sequence and are comprised of cis-acting long terminal repeats (LTRs). The minimum cis-acting LTRs are sufficient for replication and packaging of the vectors, which are then used to integrate the exogenous gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof. Buchscher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; and PCT/US94/05700).

Adeno-associated virus (AAV) vectors are also used to transduce cells with target nucleic acids, e.g., in the in vitro production of nucleic acids and peptides, and for in vivo and ex vivo applications. See, e.g., West et al. (1987) Virology 160:38-47; U.S. Pat. No. 4,797,368; WO 93/24641; Kotin (1994) Hum. Gene Ther. 5:793-801; and Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin, et al. (1984) Mol. Cell. Biol. 4:2072-2081; Hermonat et al. (1984) Proc. Natl. Acad. Sci. USA 81:6466-6470; and Samulski 0.15 et al. (1989) J. Virol. 63:3822-3828.

Recombinant adeno-associated virus vectors based on the defective and nonpathogenic parvovirus adeno-associated virus type 2 (AAV-2) are a promising nucleic acid delivery system. Exemplary AAV vectors are derived from a plasmid containing the AAV 145 bp inverted terminal repeats flanking a transgene expression cassette. Efficient transfer of nucleic acids and stable transgene delivery due to integration into the genomes of the transduced cell are key features for this vector system. Wagner et al. (1998) Lancet 351 (9117):1702-3; and Kearns et al. (1996) Gene Ther. 9:748-55. pLASN and MFG-S are examples are retroviral vectors that have been used in clinical trials. Dunbar et al. (1995) Blood 85:3048-305; Kohn et al. (1995) Nature Med. 1:1017-102; Malech et al. (1997) Proc. Natl. Acad. Sci. USA 94:12133-12138. PA317/pLASN was the first therapeutic vector used in a gene therapy trial. (Blaese et al. (1995) Science 270:475-480. Transduction efficiencies of 50% or greater have been observed for MFG-S packaged vectors. Ellem et al. (1997) Immunol Immunother. 44(1):10-20; Dranoffet al. (1997) Hum. Gene Ther. 1:111-2.

In applications for which transient expression is preferred, adenoviral-based systems are useful. Adenoviral based vectors are capable of very high transduction efficiency in many cell types and are capable of infecting, and hence delivering nucleic acid to, both dividing and non-dividing cells. With such vectors, high titers and levels of expression have been obtained. Adenovirus vectors can be produced in large quantities in a relatively simple system.

Replication-deficient recombinant adenovirus (Ad) vectors can be produced at high titer and they readily infect a number of different cell types. Most adenovirus vectors are engineered such that a transgene replaces the Ad E1 a, E1b, and/or E3 genes; the replication defector vector is propagated in human 293 cells that supply the required E1 functions in trans. Ad vectors can transduce multiple types of tissues in vivo, including non-dividing, differentiated cells such as those found in the liver, kidney and muscle. Conventional Ad vectors have a large carrying capacity for inserted DNA. An example of the use of an Ad vector in a clinical trial involved polynucleotide therapy for antitumor immunization with intramuscular injection. Sterman et al. (1998) Hum. Gene Ther. 7:1083-1089. Additional examples of the use of adenovirus vectors for nucleic acid delivery include Rosenecker et al. (1996) Infection 24:5-10; Sterman et al., supra; Welsh et al. (1995) Hum. Gene Ther. 2:205-218; Alvarez et al. (1997) Hum. Gene Ther. 5:597-613; and Topf et al. (1998) Gene Ther. 5:507-513.

Packaging cells are used to form virus particles that are capable of infecting a host cell. Such cells include 293 cells, which package adenovirus, and Ψ2 cells or PA317 cells, which package retroviruses. Viral vectors used in nucleic acid delivery are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. Missing viral functions are supplied in trans, if necessary, by the packaging cell line. For example, AAV vectors used in nucleic acid delivery typically only possess ITR sequences from the AAV genome, which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment, which preferentially inactivates adenoviruses.

In many nucleic acid delivery applications, it is desirable that the vector be delivered with a high degree of specificity to a particular tissue type. A viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751 reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., F_{ab} or Fᵥ) having specific binding affinity for virtually any chosen cellular receptor. Although the above description applies primarily to viral vectors, the same principles can be applied to non-viral vectors. Such vectors can be engineered to contain specific uptake sequences thought to favor uptake by specific target cells.

Vectors can be delivered in vivo by administration to a subject, typically by systemic administration (e.g., intravenous, intraperitoneal, intramuscular, subdermal, or intracranial infusion) or topical application, as described infra. Alternatively, vectors can be delivered to cells ex vivo, such as cells explanted from a subject (e.g., lymphocytes, bone marrow aspirates, tissue biopsy) or universal donor hematopoietic stem cells, followed by reimplantation of the cells into a subject, usually after selection for cells which have incorporated the vector.

Ex vivo cell transfection (e.g., for diagnostics, research, or for gene therapy such as via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art. See, e.g., Freshney et al., Culture of Animal Cells, Manual of Basic Technique, 3rd ed., 1994, and references cited therein, for a discussion of isolation and culture of cells from patients.

In one embodiment, hematopoietic stem cells are used in ex vivo procedures for cell transfection and nucleic acid delivery. The advantage to using stem cells is that they can be differentiated into other cell types in vitro, or can be introduced into a mammal (such as the donor of the cells) where they will engraft in the bone marrow. Methods for differentiating CD34+ stem cells in vitro into clinically important immune cell types using cytokines such a GM-CSF, IFN-γ and TNF-α are known. Inaba et al. (1992) J. Exp. Med. 176:1693-1702.

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells). See Inaba et al., supra.

Vectors (e.g., retroviruses, adenoviruses, liposomes, etc.) containing nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions described herein. See, e.g., Remington 's Pharmaceutical Sciences, 17th ed., 1989.

B. Delivery of Polypeptides

In other embodiments, fusion proteins are administered directly to target cells. In certain in vitro situations, the target cells are cultured in a medium containing one or more functional domain-ZFP fusions as described herein. In other situations, fusion proteins can be administered to cells or tissues in vivo or ex vivo.

An important factor in the administration of polypeptide compounds is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins, lipids and other compounds, which have the ability to translocate polypeptides across a cell membrane, have been described.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58. Prochiantz (1996) Curr. Opin. Neurobiol. 6:629-634. Another subsequence, the h (hydrophobic) domain of signal peptides, was found to have similar cell membrane translocation characteristics. Lin et al. (1995) J. Biol. Chem. 270:14255-14258.

Examples of peptide sequences which can be linked to a zinc finger polypeptide (or fusion containing the same) for facilitating its uptake into cells include, but are not limited to: an 11 amino acid peptide of the tat protein of HIV; a 20 residue peptide sequence which corresponds to amino acids 84-103 of the p16 protein (see Fahraeus et al. (1996) Curr. Biol. 6:84); the third helix of the 60-amino acid long homeodomain of Antennapedia (Derossi et al. (1994) J. Biol. Chem. 269:10444); the h region of a signal peptide, such as the Kaposi fibroblast growth factor (K-FGF) h region (Lin et al., supra); and the VP22 translocation domain from HSV (Elliot et al. (1997) Cell 88:223-233). Other suitable chemical moieties that provide enhanced cellular uptake can also be linked, either covalently or non-covalently, to the ZFPs.

Toxin molecules also have the ability to transport polypeptides across cell membranes. Often, such molecules (called “binary toxins”) are composed of at least two parts: a translocation or binding domain and a separate toxin domain. Typically, the translocation domain, which can optionally be a polypeptide, binds to a cellular receptor, facilitating transport of the toxin into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions. Arora et al. (1993) J. Biol. Chem. 268:3334-3341; Perelle et al. (1993) Infect. Immun. 61:5147-5156; Stenmark et al. (1991) J. Cell Biol. 113:1025-1032; Donnelly et al. (1993) Proc. Natl. Acad. Sci. USA 90:3530-3534; Carbonetti et al. (1995) Abstr. Annu. Meet. Am. Soc. Microbiol. 95:295; Sebo et al. (1995) Infect. Immun. 63:3851-3857; Klimpel et al. (1992) Proc. Natl. Acad. Sci. USA. 89:10277-10281; and Novak et al. (1992) J. Biol. Chem. 267:17186-17193.

Such subsequences can be used to translocate polypeptides, including the polypeptides as disclosed herein, across a cell membrane. This is accomplished, for example, by derivatizing the fusion polypeptide with one of these translocation sequences, or by forming an additional fusion of the translocation sequence with the fusion polypeptide. Optionally, a linker can be used to link the fusion polypeptide and the translocation sequence. Any suitable linker can be used, e.g., a peptide linker.

A suitable polypeptide can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell.

The liposome fuses with the plasma membrane, thereby releasing the compound into the cytosol. Alternatively, the liposome is phagocytosed or taken up by the cell in a transport vesicle. Once in the endosome or phagosome, the liposome is either degraded or it fuses with the membrane of the transport vesicle and releases its contents.

In current methods of drug delivery via liposomes, the liposome ultimately becomes permeable and releases the encapsulated compound at the target tissue or cell. For systemic or tissue specific delivery, this can be accomplished, for example, in a passive manner wherein the liposome bilayer is degraded over time through the action of various agents in the body. Alternatively, active drug release involves using an agent to induce a permeability change in the liposome vesicle. Liposome membranes can be constructed so that they become destabilized when the environment becomes acidic near the liposome membrane. See, e.g., Proc. Natl. Acad. Sci. USA 84:7851 (1987); Biochemistry 28:908 (1989). When liposomes are endocytosed by a target cell, for example, they become destabilized and release their contents. This destabilization is termed fusogenesis. Dioleoylphosphatidylethanolamine (DOPE) is the basis of many “fusogenic” systems.

For use with the methods and compositions disclosed herein, liposomes typically comprise a fusion polypeptide as disclosed herein, a lipid component, e.g., a neutral and/or cationic lipid, and optionally include a receptor-recognition molecule such as an antibody that binds to a predetermined cell surface receptor or ligand (e.g., an antigen). A variety of methods are available for preparing liposomes as described in, e.g.; U.S. Pat. Nos. 4,186,183; 4,217,344; 4,235,871; 4,261,975; 4,485,054; 4,501,728; 4,774,085; 4,837,028; 4,235,871; 4,261,975; 4,485,054; 4,501,728; 4,774,085; 4,837,028; 4,946,787; PCT Publication No. WO 91/17424; Szoka et al. (1980) Ann. Rev. Biophys. Bioeng. 9:467; Deamer et al. (1976) Biochim. Biophys. Acta 443:629-634; Fraley, et al. (1979) Proc. Natl. Acad. Sci. USA 76:3348-3352; Hope et al. (1985) Biochim. Biophys. Acta 812:55-65; Mayer et al. (1986) Biochim. Biophys. Acta 858:161-168; Williams et al. (1988) Proc. Natl. Acad. Sci. USA 85:242-246; Liposomes, Ostro (ed.), 1983, Chapter 1); Hope et al. (1986) Chem. Phys. Lip. 40:89; Gregoriadis, Liposome Technology (1984) and Lasic, Liposomes: from Physics to Applications (1993). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it may be desirable to target a liposome using targeting moieties that are specific to a particular cell type, tissue, and the like. Targeting of liposomes using a variety of targeting moieties (e.g., ligands, receptors, and monoclonal antibodies) has been previously described. See, e.g., U.S. Pat. Nos. 4,957,773 and 4,603,044.

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV-1) and papilloma virus antigens. Inflammation can be detected using molecules specifically recognized by surface molecules which are expressed at sites of inflammation such as integrins (e.g., VCAM-1), selectin receptors (e.g., ELAM-1) and the like.

Standard methods for coupling targeting agents to liposomes are used. These methods generally involve the incorporation into liposomes of lipid components, e.g., phosphatidylethanolamine, which can be activated for attachment of targeting agents, or incorporation of derivatized lipophilic compounds, such as lipid derivatized bleomycin. Antibody targeted liposomes can be constructed using, for instance, liposomes which incorporate protein A. See Renneisen et al. (1990) J. Biol. Chem. 265:16337-16342 and Leonetti et al. (1990) Proc. Natl. Acad. Sci. USA 87:2448-2451.

Kits

Also provided are kits for performing any of the above methods. The kits typically contains cells comprising one or more ZFP-functional domain fusion polypeptides and/or nucleic acids encoding such fusion polypeptides for use in the above methods, or components for making such cells. Some kits contain pairs of test and control cells differing in that one cell population is transformed with one or more exogenous nucleic acids encoding a ZFP-functional domain fusion protein designed to regulate expression of a molecular target or other protein within the test cells. Some kits contain a single cell type and other components that allow one to produce control and experimantal cells from that cell type. Such components can include a vector encoding a zinc finger protein or the zinc finger protein itself. Additional kits contain nucleic acids which encode one or more ZFP-functional domain fusion proteins. The kits can also contain buffers for transformation of cells, culture media for cells, and/or buffers for performing assays. Typically, the kits also contain a label indicating that the cells are to be used for screening compounds. A label includes any material such as instructions, packaging or advertising leaflet that is attached to or otherwise accompanies the other components of the kit.

Exemplary Applications and Advantages

The multiplex assays disclosed herein can be carried out in any type of cell, including prokaryotic, fungal, plant and animal cells, preferably, mammalian cells. The use of mammalian, particularly human, cells provides advantages for the screening of human therapeutics, compared to assays conducted in, e.g., yeast cells, as the compound is tested in the appropriate cellular environment.

An exemplary use for the disclosed methods and compositions is in the identification of novel ligands for nuclear receptors and/or members of signal transduction pathways. An inherent advantage is the ability to multiplex the assay within a single cell line to increase screening throughput, decrease the occurrence of false positives in the screening process, and to provide a small molecule screening platform that yields high information content on compound efficacy, specificity and toxicity in a single assay system.

The creation of a high throughput screening platform that supports multiplexing through the use of multiple ZFPs targeted to different endogenous reporter genes, each linked to a different functional domain involved in related or unrelated signal transduction pathways, toxic responses, or drug metabolism, will allow for the selection of compounds that are most efficacious and specific towards regulating their intended target(s) and exhibit the least amount of toxicity. This type of high throughput screening platform will allow for the simultaneous monitoring of compound efficacy, specificity, toxicity, and metabolism and will reduce the amount of time and cost required for secondary screening and analyses required to optimize lead compounds; thereby facilitating the identification and isolation of drug compounds with the highest therapeutic indices.

Other practical uses for the multiplex assays described herein include the identification of novel ligands for multiple drug targets using a single cell line. Several orphan receptors, (i.e., receptors with no known ligand), or several related or unrelated factors of interest can be expressed in the same cell line and targeted to different endogenous reporter genes. Novel ligands for each protein target can then be identified in a single screen of a compound library by identifying compounds that regulate the activity of each or any of the protein targets of interest. The identification of lead compounds for several drug targets in a single screen reduces the amount of time and resources required to carry out each screen individually.

The disclosed multiplex assays will also reduce the amount of false positives that result from a chemical compound regulating the expression of the reporter gene in a mechanism independent of the target factor. For example, the same functional domain or peptide can be targeted to different reporter genes, using different engineered ZFP DNA-binding domains. The criterion for a “hit” or active compound, in this type of assay is that all targeted reporter genes are regulated similarly. This provides a method by which false positives are filtered out early in the screening process. The elimination of compounds that are false positives reduces the amount of time, money, and resource that would be expended in further analyses of these compounds.

Compounds that are toxic and/or upregulate genes involved in drug metabolism can decrease drug efficacy or, worse, cause detrimental or undesired side effects. Preliminary information on drug toxicity and metabolism is achieved, according to the present disclosure, by creating fusions of ZFP binding domains with factors (or functional domains derived therefrom) involved in the recognition, catabolic breakdown, and/or removal of foreign compounds. One example is a fusion between an engineered ZFP and a xenobiotic receptor or functional fragment thereof. In this way, lead compounds can be selected based both on their ability regulate their intended target in the appropriate manner along with their inability to bind and upregulate factors involved in toxic responses or drug metabolism.

The methods and compositions disclosed herein can be used, e.g., for screening compound libraries to identify novel ligands for NHRs (nuclear hormone receptors). The examples describe cell lines expressing the ligand binding domains of ERalpha, Erbeta, TRbeta and FXR, fused to one or more engineered ZFP domains. These cell lines are used for the screening and identification of ER, TR and FXR ligands (agonists and/or antagonists) by monitoring changes in the expression of endogenous genes. Unlike natural nuclear hormone receptors, which exhibit similar DNA-binding specificities and thus suffer interference from factors that recognize similar response elements, each engineered ZFP recognizes a unique binding site. This permits efficient multiplexing for the identification of isotype-specific ligands.

Although the methods and compositions for multiplex assays have been exemplified using nuclear receptors, it will be clear to those of skill in the art that similar methods and compositions can be used to assay for drugs that target other molecules which are members of, or whose activity is regulated by, a cellular signaling cascade, or, indeed any molecule which comprises a functional domain capable of regulating gene expression.

Compounds initially identified as hits in current screening assays often regulate the activity or expression of a reporter gene through a mechanism independent of the intended target. The multiplex assays disclosed herein can be used to reduce this type of assay noise by employing fusions of a target functional domain to multiple unique ZFPs, each of which binds to a different reporter gene. By forcing the target factor to regulate more than one reporter gene, a compound will not be scored as a hit unless it modulates all the targeted reporter genes in a similar fashion.

The multiplex assays disclosed herein also permit the identification of new ligands for multiple factors in a single screen. Instead of conducting multiple screens individually examining different factors of interest, several targets of interest can be tested in a single screen. For example, simultaneous assay of a target molecule and related proteins (e.g., family members, isotypes, splice variants) and/or factors involved in toxic responses (e.g., xenobiotic receptors), and/or factors involved in drug metabolism (e.g., MDRs, antiporters), using the methods and compositions disclosed herein, can provide additional information on compound specificity, as well as preliminary information on drug toxicology and metabolism.

### EXAMPLES

The following examples are presented as illustrative of, but not limiting, the claimed subject matter.

### Example 1

### Material and Methods

Cell culture and transient transfections—HEK293 cells were grown in Dulbecco's modified Eagle's medium (DMEM) (Invitrogen, Carlsbad, Calif.) supplemented with 10% fetal bovine serum (FBS) filtered through charcoal-dextran (Hyclone). All cells were maintained at 37° C. in an atmosphere of 5% CO₂. HEK293 cells were transfected using LipofectAMINE 2000 Reagent (Invitrogen) in Opti-MEM I reduced serum medium according to the manufacturer's protocol. Cells were treated with the appropriate ligand for 24 hours before harvesting for RNA isolation.

Ligand storage and treatment—17alpha-estradiol; 17beta-estradiol; 3,3′,5-Triiodo-L-thyronine (T3); and Chenodeoxycholic acid (CDCA) were obtained from Sigma-Aldrich Corp (St. Louis, Mo.) and resuspended in Dimethyl sulfoxide (DMSO). 17alpha estradiol was maintained at a stock concentration of 10 mM, 17beta-estradiol and T3 were maintained at a stock concentration of 1 mM, and CDCA was maintained at a stock concentration of 100 uM. Stocks were diluted in DMSO to 1000× and/or added directly to cells for 24 hours at 37° C.

Total RNA isolation and quantitative RT-PCR—Total RNA was isolated from HEK293 cells using the High Pure Isolation Kit (Roche Molecular Biochemicals, Indianapolis, Ind.) and 25 ng of total RNA from each sample was subjected to real time quantitative RT-PCR to analyze endogenous gene expression, using TaqMan® assays. Reactions were carried out on an ABI 7700 SDS machine (Perkin-Elmer Life Sciences, Foster City, Calif.) under the following conditions. The reverse transcription reaction was performed at 48° C. for 30 minutes with MultiScribe reverse transcriptase (Perkin-Elmer Life Sciences), followed by a 10-minute denaturation step at 95° C. Polymerase chain reaction (PCR) was carried out with AmpliGold DNA polymerase (Perkin-Elmer Life Sciences) for 40 cycles at 95° C. for 15 seconds and 60° C. for 1 minute. Results were analyzed using the SDS version 1.7 software. The expression of each endogenous gene, Kip2, GRP, and AnnexinA8, was normalized to the expression of the human GAPDH gene.

Sequences of the oligonucleotides used as probes and primers in the real-time PCR analysis are given in Table 1. For analysis of AnnexinA8 and Kip2 mRNAs, final concentrations of 0.9 uM forward and reverse primers, and 0.1 uM probe were used in the amplification reaction. For analysis of GRP mRNA, final concentrations of 0.3 uM forward primer, 0.9 uM reverse primer and 0.1 uM probe were used in the amplification reaction. For analysis of GAPDH mRNA, final concentrations of 0.1 uM forward primer, 0.3 uM reverse primer and 0.1 uM probe were used in the amplification reaction.

TABLE 1
Probe and primer sequences for RNA analysis

| | | | |
|---|---|---|---|
| Gene | Oligonucleotide | Sequence | SEQ ID NO |

| | | | |
|---|---|---|---|
| AnxA8 | Forward primer | ACGCGCAGTGCCACTCA | 10 |
| | Reverse primer | TGATGCTGTCCTCAATGCTCTT | 11 |
| | Probe | CTGAGAGTGTTTGAAGAGTATGAGAAAATTGCCAA | 12 |
| Kip2 | Forward primer | GCGCGGCGATCAAGAA | 13 |
| | Reverse primer | ACATCGCCCGACGACTTC | 14 |
| | Probe | CCGGGCCTCTGATCTCCGATTTCT | 15 |
| GRP | Forward primer | AGGCCCTGGGCAATCAG | 16 |
| | Reverse primer | CAACTTTGCCTTTTGAACCTACATC | 17 |
| | Probe | AGCCTTCGTGGGATTCAGAGGATAGCAG | 18 |
| GAPDH | Forward primer | CCATGTTCGTCATGGGTGTGA | 19 |
| | Reverse primer | CATGGACTGTGGTCATGAGT | 20 |
| | Probe | TCCTGCACCACCAACTGCTTAGCA | 21 |

### Example 2

### Expression Vectors

Mammalian expression vectors encoding engineered ZFPs fused to the ligand binding domains of Nuclear Hormone Receptors were derived from the plasmid pcDNA-NKF, previously described in WO 00/41566. Briefly, the pcDNA-NKF vector was constructed by digesting the plasmid pcDNA3.1(+) (Invitrogen) with HindIII and BamHI, filling-in the protruding ends and re-ligating. This plasmid was further modified by inserting a fragment between its EcoRI and XhoI sites containing the following:

(1) a segment from EcoRI to KpnI containing the Kozak translation initiation sequence (including the initiation codon) and the SV40 nuclear localization sequence, altogether comprising the DNA sequence

GAATTCGCTAGCGCCACCATGGCCCCCAAGAAGAAGAGGAAGGTGGG AATCCATGGGGTAC(SEQ ID NO: 22), where the EcoRI and KpnI sites are underlined; and

(2) a segment from KpnI to XhoI containing a BamHI site, the KRAB-A box from KOX1 (amino acid coordinates 11-53 in Thiesen et al. (1990) New Biologist 2:363-374), the FLAG epitope (Kodak/IBI), and a HindIII site, altogether comprising the sequence GGTACCCGGGGATCCCGGACACTGGTGACCTTCAAGGATGTATTTGTGGACT TCACCAGGGAGGAGTGGAAGCTGCTGGACACTGCTCAGCAGATCGTGTACAG AAATGTGATGCTGGAGAACTATAAGAACCTGGTTTCCTTGGGCAGCGACTAC AAGGACGACGATGACAAGTAAGCTTCTCGAG(SEQ ID NO: 23), where the KpnI, BamHI and XhoI sites are underlined.

Vectors encoding a targeted ZFP binding domain fused to the NLS, KRAB and FLAG domains were constructed by inserting a KpnI-BamHI cassette containing the ZFP-encoding sequences into KpnI/BamHI digested pcDNA-NKF. These constructs were named pcDNA3-modZFP(#)-NKF, where “#” denotes the ZFP binding domain (see Tables 2 and 3).

### Example 3

### Design of ZFPs that Bind the Endogenous Kip2, GRP and anxA8 Genes

ZFP binding domains were designed, fused to the VP16 transcriptional activation domain, and tested for their ability to regulate the expression of the human genes Kip2, Gastrin-releasing peptide (GRP), and AnnexinA8 (AnxA8). The methods for the design and synthesis of zinc finger proteins able to bind to preselected sites disclosed in co-owned U.S. Pat. No. 6,453,242; WO 00/41566 and PCT/US01/43568 were used to generate three constructs: one encoding a ZFP that bound to the human Kip2 gene, one encoding a ZFP that bound to the human GRP gene and one encoding a ZFP that bound to the human anxA8 gene. Target genes, binding sites and sequences of the recognition regions of the zinc fingers of these proteins are given in Table 2.

TABLE 2
Designed zinc finger protein binding domains

| | | | | | |
|---|---|---|---|---|---|
| ZFP# | target | binding site | F1 sequence* | F2 sequence* | F3 sequence* |

| | | | | | |
|---|---|---|---|---|---|
| 734 | kip2 | GGGGCTGGGT | RSDHLAR | QSSDLSR | RSDHLSR |
| | | (SEQ ID NO:24) | (SEQ ID NO:25) | (SEQ ID NO:26) | (SEQ ID NO:27) |
| 1727 | GRP | GGTGGGGAGG | RSDNLAR | RSDHLTR | TSGHLVR |
| | | (SEQ ID NO:28) | (SEQ ID NO:29) | (SEQ ID NO:30) | (SEQ ID NO:31) |
| 757 | anxA8 | CGGGCGGCTG | QSSDLRR | RSDELQR | RSDHLRE |
| | | (SEQ ID NO:32) | (SEQ ID NO:33) | (SEQ ID NO:34) | (SEQ ID NO:35) |

*The amino acid sequences shown are those of amino acids −1 through +6 (with respect to the start of the alpha-helical portion of the zinc finger) and are given in the one-letter code

Sequences encoding the ZFP binding domains shown in Table 2 were individually fused to sequences encoding a VP16 transcriptional activation domain. The constructs were transfected into HEK 293 cells, and expression of the encoded protein resulted in activation of expression of the appropriate gene (i.e., the ZPF734-VP16 fusion activated kip2 gene expression, the ZPF1727-VP16 fusion activated GRP gene expression, and the ZPF757-VP16 fusion activated anxA8 gene expression). Having confirmed the ability of these ZFP binding domains to specifically recognize, and regulate expression of, their intended endogenous target genes, they were fused to ligand binding domains of different nuclear receptors, as described in the following examples.

### Example 4

### Generation of a Construct Encoding a Fusion Between the FXR Receptor Ligand Binding Domain and a ZFP Targeted to the Kip2 Gene

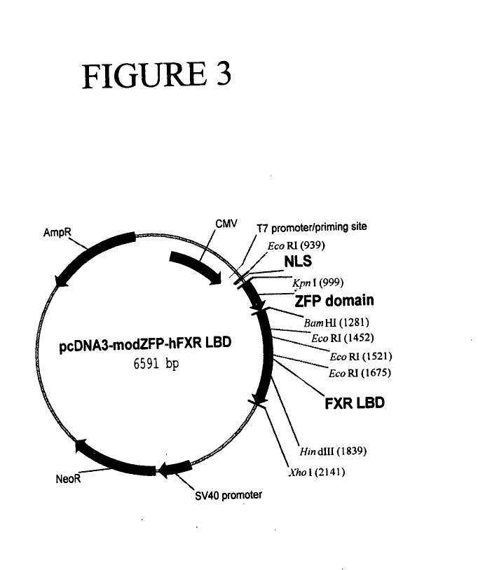

A plasmid encoding the ZFP734 binding domain fused to the ligand binding domain of the human Farnesoid-X-receptor (FXR) was constructed as follows. The ligand binding domain of human FXR (amino acids 222-472) was PCR amplified with the Platinum(R) Taq DNA Polymerase High Fidelity kit (Invitrogen) from cDNA generated from 5 ug of total RNA from human liver tissue (BD Biosciences Clontech). The cDNA synthesis reactions were carried using the SUPERSCRIPT™ Choice System for cDNA Synthesis kit (Invitrogen) according to the manufacturer's protocol. An 869 bp fragment was isolated, and BamHI and XhoI restriction sites were engineered onto the 5′- and 3′-termini, respectively. This fragment was cleaved with BamHI and XhoI and ligated into the pcDNA3-modZFP(734)-NKF vector, encoding the ZFP734 domain (Table 2). This results in the removal of the KRAB domain from pcDNA3-modZFP(734)-NKF and its replacement by the ligand binding domain of FXR, thereby fusing the FXR ligand binding domain to the ZFP734 domain. This construct was named pcDNA3-modZFP-hFXR LBD (734-FXR LBD). See FIG. 3.

### Example 5

### Generation of a Construct Encoding a Fusion Between the Thyroid Hormone Receptor Beta Ligand Binding Domain and a ZFP Targeted to the GRP Gene

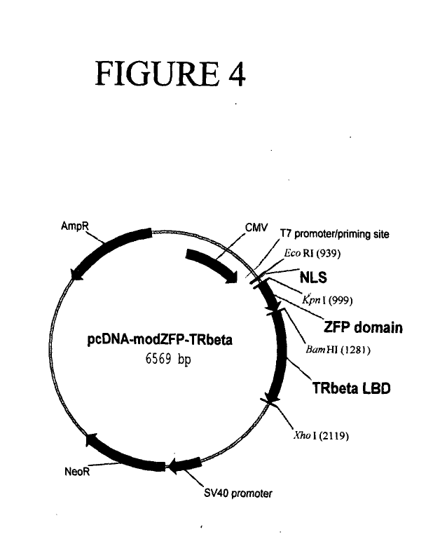

A plasmid encoding the ZFP1727 binding domain fused to the ligand binding domain of human Thyroid hormone receptor, beta (TRβ) was constructed as follows. The ligand binding domain of human TRβ (amino acids 187-456) was PCR amplified from cDNA generated from 5 ug of total RNA from human thyroid tissue (BD BioSciences Clonetech), as described above. This generated an 849 bp fragment with BamHI and XhoI sites on its 5′- and 3′-termini, respectively. This fragment was cleaved with BamHI and XhoI and ligated into pcDNA3-modZFP(1727)-NKF vector, encoding the ZFP1727 domain (Table 2). This results in the removal of the KRAB domain and its replacement by the ligand binding domain of TRβ. This construct was named pcDNA3-modZFP-TRbeta (1727-TRb). See FIG. 4.

### Example 6

### Generation of a Construct Encoding a Fusion Between the Estrogen Receptor Alpha Ligand Binding Domain and a ZFP Targeted to the anxA8 Gene

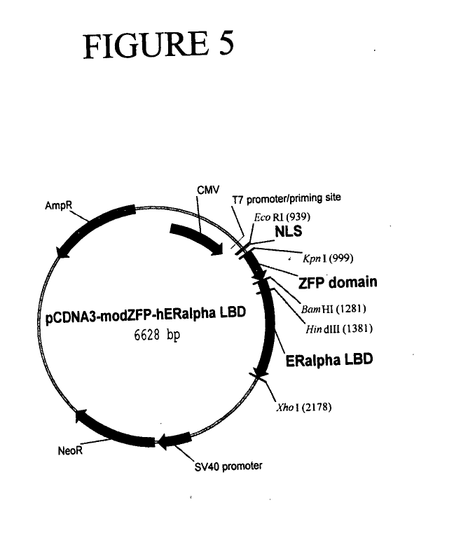

A plasmid encoding the ZFP757 binding domain fused to the ligand binding domain of human Estrogen receptor alpha (ERα) was constructed as follows. The ligand binding domain of human ERα (amino acids 307-595) was PCR amplified from cDNA generated from 5 ug of total RNA from human ovarian tissue (BD BioSciences Clonetech), as described above. A 903 bp fragment, with BamHI and XhoI restriction sites on the 5′- and 3′-termini, respectively, was obtained. This fragment was cleaved with BamHI and XhoI and ligated into pcDNA3-modZFP(757)-NVF vector, encoding the ZFP757 domain (Table 2), also cleaved with BamHI and XhoI. This results in the removal of the KRAB domain and its replacement by the ligand binding domain of ERα. This construct was named pcDNA3-modZFP-hERalpha LBD (757-ERa). See FIG. 5.

### Example 7

### Independent Regulation of the Kip2, GRP and anxA8 Genes by ZFP-Nuclear Receptor Fusions in a Single Cell Population

This example demonstrates a multiplex assay in which the activity of three different nuclear receptors is assayed in a single cell population. Cells were transfected with three plasmids: each encoding a fusion of distinct nuclear receptor with a ZFP targeted to a unique endogenous cellular gene. Thus, the readout for activity of each receptor is expression of a distinct endogenous cellular gene, allowing the receptors to be assayed simultaneously.

HEK293 cells were plated into 6-well dishes and, in each well, the cells were co-transfected with a mixture of 0.5 ug of pcDNA3-modZFP-hFXR LBD (734-FXR LBD), 0.3 ug pcDNA3-modZFP-TRbeta (1727-TRb), and 0.3 ug pcDNA3-modZFP-hERalpha LBD (757-ERa). See Examples 4-6, above, (and FIGS. 3-5) for the structures of these plasmids. In separate wells, cells were treated for 24 hours with DMSO (negative control), 100 nM 17beta-estradiol, 100 nM T3, or 100 nM CDCA, and total RNA was harvested as described in Example 1. Real-time PCR (TaqMan®) analysis was performed, as described in Example 1, to quantitate the expression of each endogenous gene target (Kip2, GRP, and AnxA8) in response to each compound. The expression of each gene was normalized to that of GAPDH, and fold changes were determined by dividing the normalized expression in the presence of the compound by the expression in the cells treated with DMSO.

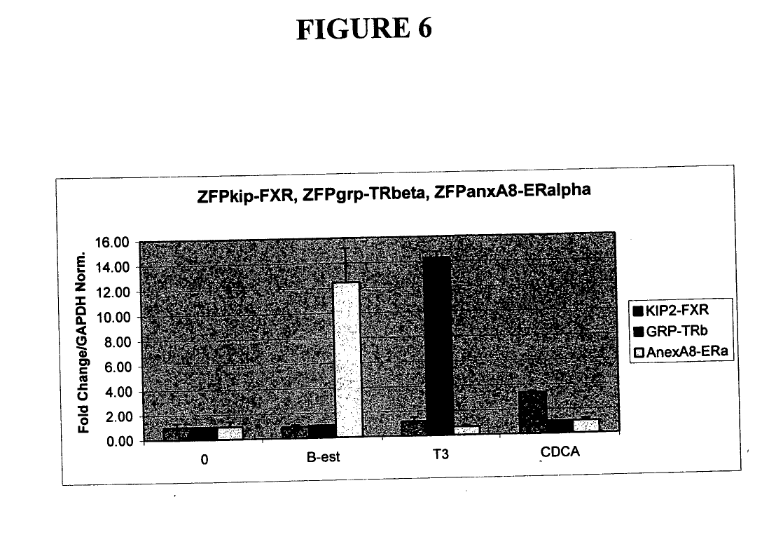

The results are shown in FIG. 6. In cells treated with 17beta-estradiol, the activity of the ZFP 757 (ZFPanxA8)/ERalpha fusion protein was induced, and expression of the AnnexinA8 gene increased by approximately 12-fold, compared to untreated cells. Transfected cells treated with T3 showed a 14-fold upregulation of the 1727-TRbeta-targeted GRP gene. Cells treated with the FXR ligand, CDCA, showed roughly a 3.5-fold increase in Kip2 expression when compared with the untreated sample. These results demonstrate that distinct functional domains, each linked to a different ZFP binding domain, can be expressed and targeted to different endogenous genes in a single cell, and that changes in the expression of the targeted endogenous genes reflect the ability of compounds to regulate the activity of the functional domains.

### Example 8

### Generation of Stable Cell Lines Expressing 993(ZFPkip2)-hERalpha

This example describes the preparation of a construct encoding a Kip2-targeted ZFP fused to the ligand-binding domain of the human estrogen receptor alpha (hERα) and the generation of a cell line in which this construct is stably integrated into the genome.

Sequences encoding the ligand binding domain of human ERα were isolated from the pcDNA3-modZFP-hERalpha LBD (757-ERa) vector (Example 6) by cleavage with BamHI and XhoI, and ligated into the pcDNA3-modZFP(993)-NKF vector, encoding the ZFP993 domain (constructed as described in Example 2). The amino sequences of the zinc finger recognition regions of the ZFP 993 protein, as well as the DNA target sequence, are given in Table 3. This construct was named pcDNA3-modZFP-hERalpha LBD (993).

TABLE 3
Designed zinc finger protein binding domains

| | | | | | |
|---|---|---|---|---|---|
| ZFP# | target | binding site | F1 sequence* | F2 sequence* | F3 sequence* |

| | | | | | |
|---|---|---|---|---|---|
| 993 | kip2 | GGGGCTGGGT | RSDHLAR | TSGELVR | RSDHLSR |
| | | (SEQ ID NO:36) | (SEQ ID NO:37) | (SEQ ID NO:38) | (SEQ ID NO:39) |

*The amino acid sequences shown are those of amino acids −1 through +6 (with respect to the start of the alpha-helical portion of the zinc finger) and are given in the one-letter code

HEK293 cells were plated into 6-well dishes at 50% confluence, and two wells were each transfected with 0.9 ug of pcDNA3-modZFP-hERalpha LBD plasmid, expressing 993-hERalpha. The cells were allowed to recover for 48 hours, and then both wells were combined and split into 10×15-cm² dishes in selective medium; i.e., standard medium supplemented with 400 ug/ml G418 (Invitrogen). The medium was changed every 3 days, and after 10 days single colonies were isolated and further expanded in T-25 flasks. Each clonal line was tested individually by the addition of 100 nM 17-beta-estradiol. The cell lines with the highest activation of the endogenous Kip2 gene in response to 17-beta-estradiol were maintained and made into frozen stocks. One of these lines was selected for further experiments.

### Example 9

### Ligand-Mediated Regulation of Multiple Reporter Genes in a Stable Cell Line

The cell line described in the previous example, which contains a stably-integrated construct expressing a Kip2-targeted DNA-binding domain fused to ERalpha, was transiently transfected with a plasmid encoding a GRP-targeted ZFP binding domain fused to the ligand binding domain of TRβ (pcDNA3-modZFP-TRbeta (1727-TRb), see Example 5). Transfections were carried out in 12-well dishes; the cells in each well being transfected with 0.5 ug of pcDNA-modZFP-TRbeta, expressing 1727-TRbeta (ZFPGRP). Twenty-four hours after transfection, one set of cells was treated with a serial dilution of the ER ligand, 17-beta-estradiol, and another set of cells was treated with the TR ligand, T3. Each titration series ranged from 10⁻⁵ M to 10⁻¹¹ M, final concentration of ligand. After 24 hours, cells were harvested and total RNA was isolated. Real-time PCR analysis was performed on each sample to quantitate changes in the expression of Kip2 and GRP, normalized to GAPDH.

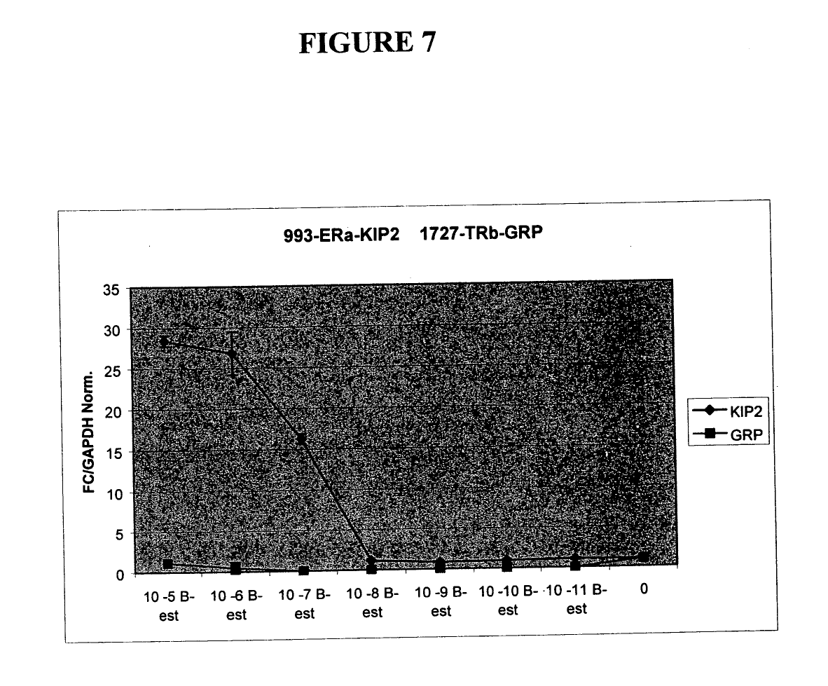

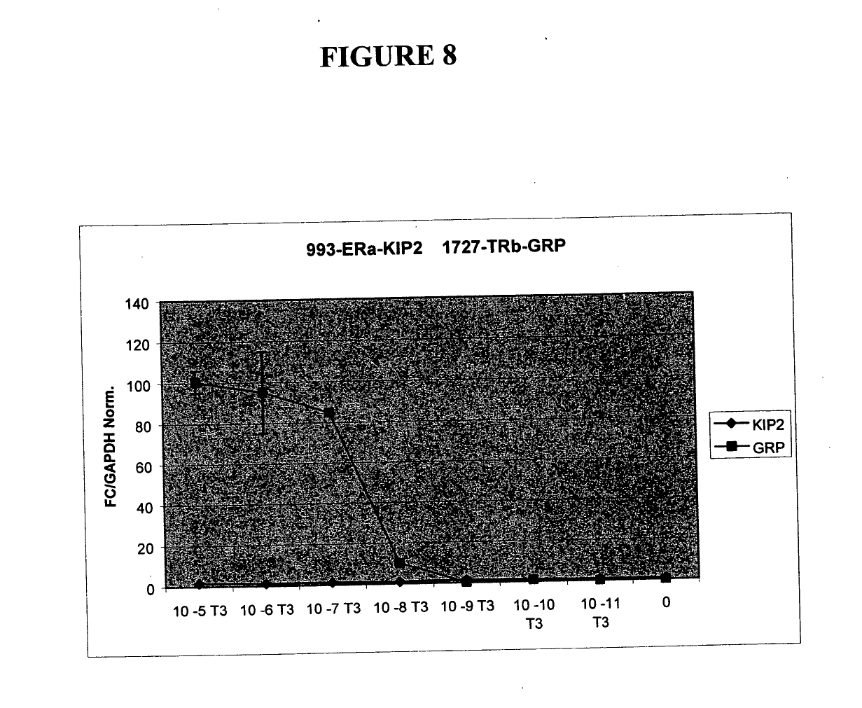

Cells treated with 17-beta-estradiol showed a dose-dependent increase in Kip2 expression, consistent with the normal response of the endogenous ERalpha receptor to 17-beta-estradiol (FIG. 7). Expression of the GRP gene is not altered by treatment with 17beta-estradiol (FIG. 7). Conversely, in cells treated with a series of T3 concentrations, expression of GRP is regulated by T3 in a dose-dependent manner (FIG. 8), consistent with the normal response of endogenous TRbeta to T3. No change in the expression of Kip2 is observed at any concentration of T3 (FIG. 8). These results demonstrate that physiological, dose-dependent regulation of ERα and TRβ can be obtained in a single cell population and assayed by expression of endogenous genes in that cell population. Furthermore, they show the feasibility of conducting such multiplex assays in stable cell lines.

### Example 10

### Generation of a Construct Encoding a Fusion Protein Between the Estrogen Receptor Beta Ligand Binding Domain and a ZFP Targeted to the GRP Gene

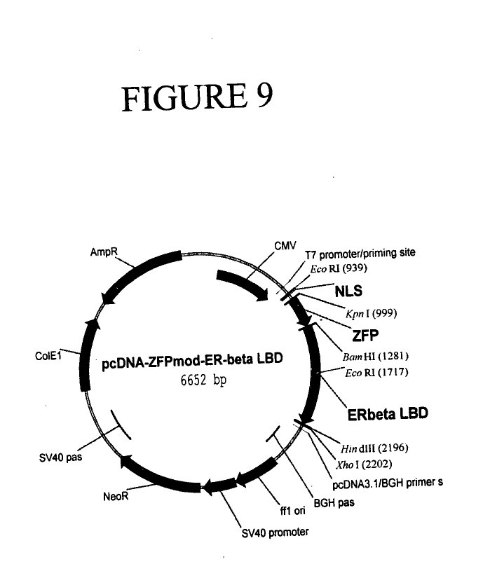

A plasmid encoding the ZFP1727 binding domain fused to the ligand binding domain of human estrogen receptor beta (ERβ) was constructed as follows. The ligand binding domain of human ERβ (amino acids 229-530) was isolated in a manner similar to that described for ERα, by PCR amplification from human ovarian cDNA, as described above. A 921 bp fragment was obtained, and BamHI and HindIII restriction sites were engineered onto the 5′- and 3′termini, respectively. This fragment was cleaved with BamHI and HindIII and ligated into the pcDNA3-modZFP(1727)-NVF vector, encoding the ZFP1727 domain (Table 2). This construct was named pcDNA3-modZFP-ERbeta (1727-ERb), and encodes a GRP-targeted ZFP fused to the ligand binding domain of ERβ. See FIG. 9.

### Example 11

### Generation of a Stable Cell Line Expressing Two ZFP-ligand Binding Domain Fusions

Retroviral Vectors. Retroviral vectors for 993-ERα (Example 8) and 1727-ERβ (Example 10) constructs were obtained by subcloning each into a modified CMV-pSIR vector (Clontech), a self-inactivating retroviral vector which lacks U3 enhancers in the 3′ long terminal repeat (LTR) such that, upon proviral integration no enhancer remains in the provirus. An internal CMV promoter controls transgene expression in the modified vector. The 993-ERα and 1727-ERβ-encoding sequences were subcloned into a multiple cloning site that lies downstream of a tetracycline-inducible CMV promoter that contains two copies of the tet operator 2 (tetO₂) (TREx Invitrogen). Each ZFP-TF virus was marked with a different antibiotic resistance marker: neomycin for the 993-ERα and blasticidin for the 1727-ERβ.

Packaging and Transduction of ZFP-TF Containing Retroviral Vectors.

Amphotropic viruses were produced by using the high-titer 293 Phoenix packaging cell line derived by Nolan (Stanford Univ.). Briefly, 10 ug of plasmid DNA for each retroviral construct and 50 ug of Lipofectamine 2000 (GIBCO-BRL-Invitrogen) were used to transfect 5×10⁶ cells that had been seeded in 10 cm dishes. The transfection mix was removed after eight hours and replaced with fresh growth medium, then the cells were allowed to incubate an additional 48-72 hours at 37° C. At that time the medium containing the virus particles was harvested, filtered through a 45 uM filter, and frozen at −80° C.

For transductions, HEK293 cells were plated at a density of 3×10 ⁵ cells/well of a 6-well culture plate. At 24 hours after plating, the cells were infected by two exposures (2 ml) of the 993 ERα-Neo^{r} viral supernatant to 4 ug/ml polybrene. After 48 hours the cells were split and plated in 15 cm dishes at a low density and selected with 400 μg/ml G418 for 10 days. Fifty-five colonies of Neo^{r} clones were isolated and amplified. The selected clones were analyzed by TaqMan for an increase in the level of mRNA of the kip2 reporter gene. Four clones that were identified as positive for activation of the reporter gene were expanded and plated for infection with the 1727-ERβ-blasticidin virus. The transduction protocol was the same as above. After 48 hours the cells were split and plated in 15 cm dishes at a low density and selected with 5 μg ml blasticidin for 10 days. Twenty-two doubly-resistant clones (resistant to G418 and blasticidin) were isolated, expanded and tested for ligand-specific activation of the reporter genes. Each clone was treated with 100 nM 17beta-estradiol for 24 hours to test for induction of the reporter genes and total RNA was harvested. RNA from each clone was analyzed for expression of 993-ERα, 1727-ERβ, Kip2, and GRP by quantitative RT-PCR, using TaqMan assays. Cell lines that exhibited expression of ERα, ERβ, and induced expression of the two endogenous reporter genes, Kip2 and GRP, were identified and maintained.

### Example 12

### Regulation of Two Reporter Genes in a Stable Cell Line Expressing Two ZFP-Ligand Binding Domain Fusions

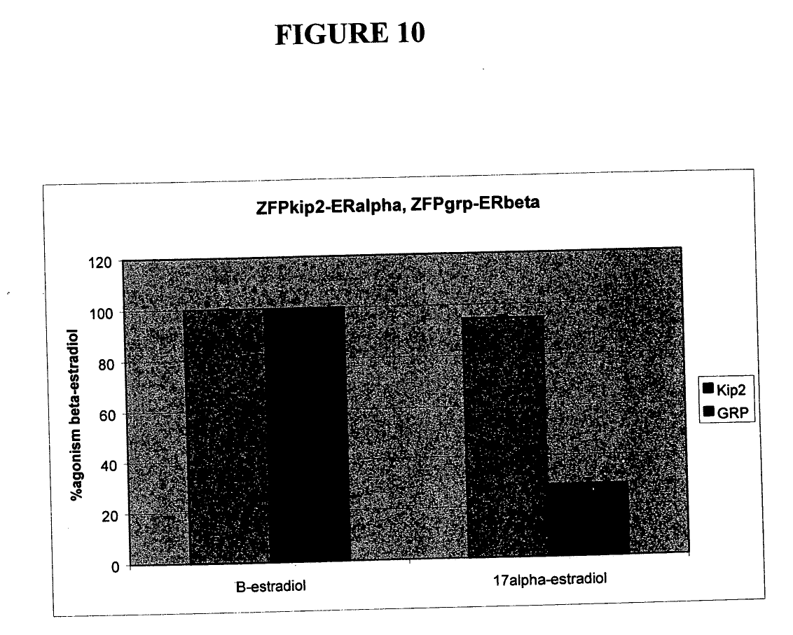

The cell line described above, which stably expresses a Kip2-targeted DNA-binding domain fused to the ERα ligand binding domain, and a GRP-targeted DNA-binding domain fused to the ERβ ligand binding domain, was tested by seeding a 12-well dish overnight and treating the cells with DMSO, 100 nM 17beta-estradiol, or 1 uM 17alpha-estradiol for 24 hours. While β-estradiol is known to activate ERα and ERβ to similar extents, α-estradiol preferentially activates ERα. Barkham et al. (1998) Molecular Pharmacology, 54:105-112. Total RNA was harvested from each well and subjected to TaqMan analysis to determine the relative expression levels of each of the targeted endogenous reporter genes. Expression of the Kip2 and GRP genes were measured and normalized to the human GAPDH gene. In order to normalize for the relative expression difference of the two endogenous reporter genes, activation of kip2 and GRP by 17beta-estradiol was set to 100%. Activation of the two endogenous genes by 17alpha-estradiol was expressed as a percentage of the activation seen with 17beta-estradiol. The results (FIG. 10) show that kip2 mRNA levels in cells treated with 17alpha-estradiol were 94.5% of those in cells that were treated with 17beta-estradiol; while GRP mRNA levels in cells treated with 17alpha-estradiol were only 28.3% of those measured in cells that had been treated with 17beta estradiol. Thus, 17alpha-estradiol preferentially stimulates ERα (as measured by expression of Kip2), compared to ERβ (as measured by GRP mRNA levels). The preferential response of the ZFP-ERα fusion to 17alpha-estradiol, compared to the ZFP-ERβ fusion, mimics the response of the natural receptors, demonstrating the usefulness of the multiplex screening assay for identifying isotype-specific compounds.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method for screening a compound, wherein the method comprises:
(a) contacting the compound with a cell, wherein the cell comprises:
(i) a first polynucleotide encoding a protein comprising a fusion between a first functional domain and a first engineered zinc finger protein targeted to a first endogenous cellular gene; and
(ii) a second polynucleotide encoding a protein comprising a fusion between a second functional domain and a second engineered zinc finger protein targeted to a second endogenous cellular gene; and
(b) measuring expression of the first and second endogenous genes.

2. The method of claim 1, wherein the first functional domain is a drug target or a functional fragment thereof.

3. The method of claim 2, wherein the second functional domain is a drug target or functional fragment thereof.

4. The method of claim 3, wherein the first and second functional domains are from the same drug target.

5. The method of claim 3, wherein the first and second functional domains are from different drug targets.

6. The method of claim 2, wherein the second functional domain is a protein related to the drug target or a functional fragment thereof.

7. The method of claim 2, wherein the second functional domain is a xenobiotic receptor or a functional fragment thereof.

8. The method of claim 2, wherein the second functional domain is a molecule involved in drug metabolism or a functional fragment thereof.

9. The method of claim 1, wherein the first functional domain is a hormone receptor, an orphan receptor, or a functional fragment thereof.

10. The method of claim 1, wherein the first polynucleotide is stably integrated into the chromosome of the cell.

11. The method of claim 10, wherein the second polynucleotide is stably integrated into the chromosome of the cell.

12. The method of claim 1, wherein the cell is a mammalian cell.

13. The method of claim 1, wherein expression of the endogenous genes is measured by assaying RNA levels.

14. The method of claim 1, wherein expression of the endogenous genes is measured by assaying protein levels.

15. The method of claim 1, wherein expression of the endogenous genes is measured by assaying enzymatic activity of the gene products.

16. The method of claim 1, wherein expression of the first endogenous gene is activated by the first functional domain.

17. The method of claim 1, wherein expression of the first endogenous gene is repressed by the first functional domain.

18. The method of claim 1, wherein the compound is screened for specificity.

19. The method of claim 1, wherein the compound is screened for toxicity.

20. The method of claim 1, wherein the compound is screened for its metabolic properties.

21. A cell comprising:
(a) a first polynucleotide encoding a protein comprising a fusion between a first functional domain and a first engineered zinc finger protein targeted to a first endogenous cellular gene; and
(b) a second polynucleotide encoding a protein comprising a fusion between a second functional domain and a second engineered zinc finger protein targeted to a second endogenous cellular gene.

22. The cell of claim 21, wherein the first functional domain is a drug target or a functional fragment thereof.

23. The cell of claim 22, wherein the second functional domain is a drug target or functional fragment thereof.

24. The cell of claim 23, wherein the first and second functional domains are from the same drug target.

25. The method of claim 23, wherein the first and second functional domains are from different drug targets.

26. The cell of claim 22, wherein the second functional domain is a protein related to the drug target or a functional fragment thereof.

27. The cell of claim 22, wherein the second functional domain is a xenobiotic receptor or a functional fragment thereof.

28. The cell of claim 22, wherein the second functional domain is a molecule involved in drug metabolism or a functional fragment thereof.

29. The cell of claim 21, wherein the first functional domain is a hormone receptor, an orphan receptor, or a functional fragment thereof.

30. The cell of claim 21, wherein the first polynucleotide is stably integrated into the chromosome of the cell.

31. The cell of claim 30, wherein the second polynucleotide is stably integrated into the chromosome of the cell.

32. The cell of claim 21, wherein the cell is a mammalian cell.

33. The cell of claim 21, further comprising a third polynucleotide encoding a protein comprising a fusion between a third functional domain and a third engineered zinc finger protein targeted to a third endogenous cellular gene.

34. The cell of claim 33, further comprising a fourth polynucleotide encoding a protein comprising a fusion between a fourth functional domain and a fourth engineered zinc finger protein targeted to a fourth endogenous cellular gene.

35. The cell of claim 34, further comprising a fifth polynucleotide encoding a protein comprising a fusion between a fifth functional domain and a fifth engineered zinc finger protein targeted to a fifth endogenous cellular gene.
